(19) United States
(12) Patent Application Publication
(10) Pub. No.: US 2021/0289773 A1
(43) Pub. Date: Sep. 23, 2021

(54) POLY-LYSINE DERIVATIVE AND ITS USE IN SOLID-BASED COMPOSITIONS

(71) Applicant: BASF SE, Ludwigshafen am Rhein (DE)

(72) Inventors: Markus Kalt, Ludwigshafen (DE); Helmut Witteler, Ludwigshafen (DE); Tina Schroeder-Grimonpot, Ludwigshafen (DE); Markus Meise, Ludwigshafen (DE); Hans-Peter Seelmann-Eggebert, Limburgerhof (DE); Frank Klippel, Ludwigshafen (DE)

(21) Appl. No.: 16/612,202

(22) PCT Filed: Apr. 17, 2018
(86) PCT No.: PCT/EP2018/059702

§ 371 Date: Nov. 8, 2019

(30) Foreign Application Priority Data
May 12, 2017 (EP) 17170900.9
Sep. 25, 2017 (EP) 17192942.5
Dec. 21, 2017 (EP) 17209244.7

(51) Int. Cl. A01N 25/10; A01N 43/54; A01N 25/14; C08G 69/10; C08G 69/04; C08G 69/48

(52) U.S. Cl. CPC A01N 25/10; A01N 43/54; C08G 69/48; C08G 69/10; C08G 69/04; A01N 25/14

(57) ABSTRACT

Described herein is a poly-lysine derivative obtained by a process including heating an aqueous lysine solution to boiling, increasing a temperature of the aqueous lysine solution to a reaction temperature in the range of about 105° C. to about 180° C., and keeping the reaction temperature in the range of about 105° C. to about 180° C. until (i) a melt viscosity of the reaction mixture is in the range of about 350 mPa*s to about 6,500 mPa*s, and (ii) an amine number in the range of about 100 mg KOH/g to about 500 mg KOH/g is achieved. The process also includes adding alkyl-carboxylic acid or alkenyl-carboxylic acid in amounts of 2.5 mol % to 10 mol %, and increasing or keeping the reaction temperature in the range of about 105° C. to about 180° C. until number of free alkyl-carboxylic acid or alkenyl-carboxylic acid is ≤9% by weight.

The present invention relates to a poly-lysine derivative selected from poly-lysine functionalized with fatty acid(s). The present invention also relates to the process of production of said polylysine derivative. The present invention further relates to solid-based compositions comprising said poly-lysine derivative. The invention furthermore relates to a process for the preparation of a solid-based composition; to the use of this composition for wetting and/or dispersing solid particles.

The present invention comprises combinations of preferred features with other preferred features.

There is a continuous need for dispersing agents for several applications in industry that preferably have wetting properties. A dispersant or dispersing agent means a substance that improves separation of solid particles and/or prevents settling or clumping of said solid particles. A wetting agent means a substance that lowers surface tension between a liquid phase and solid particles.

Applications include but are not limited to (a) dispersant used as additive for lubricating oils used in automotive engines to prevent the accumulation of varnish like deposits on individual parts of an engine, (b) dispersant used in gasoline to prevent buildup of gummy residues, (c) dispersant used to prevent formation of biofilms by dispersing microbial slime e.g. in production plants, (d) dispersant used in cleaning for dispersing particles one removed from a surface, (e) dispersant used as aid in breaking up solids as fine particles during oil drilling, (f) dispersant used to prevent unwanted deposits of inorganic and/or organic particles e.g. in production plants, (g) dispersants used in liquid coatings to disperse and/or keep dispersed pigment particles through manufacture, storage, application, and film formation.

The object of the current invention was to provide a polymer having dispersing properties preferably additionally having wetting properties.

The problem was solved by providing a poly-lysine derivative, wherein said poly-lysine derivative is obtained by a process comprising the steps of
(a) heating an aqueous lysine solution to boiling
(b) increasing temperature of the aqueous lysine solution to a reaction temperature in the range of about 105° C. to about 180° C.
(c) keep the reaction temperature in the range of about 105° C. to about 180° C. until
   i. melt viscosity of the reaction mixture in the range of about 350 mPa*s to about 6,500 mPa*s when measured at 160° C. and
   ii. an amine number in the range of about 100 mg KOH/g to about 500 mg KOH/g is achieved
(d) optionally, the vacuum applied is released
(e) add alkyl-carboxylic acid or alkenyl-carboxylic acid in amounts of 2.5 mol % to 10 mol %, relative to the theoretical amount of poly-lysine comprised in the reaction mixture
(f) increase or keep the reaction temperature in the range of about 105° C. to about 180° C. until number of free alkyl-carboxylic acid or alkenyl-carboxylic acid is 9% by weight, relative to the total weight of the reaction mixture,
   wherein vacuum is applied either in step (a), (b) and/or (c) and water is removed continuously during the whole process.

In one embodiment, the poly-lysine derivative obtained by the inventive process is further processed by the following additional steps:
(g) vacuum applied is released and the temperature within the reaction mixture is reduced to about 150° C. to about 100° C.
(h) water is added to yield a poly-lysine derivative solution comprising about 60 parts polylysine derivative and about 40 parts water.

In one embodiment, the poly-lysine is modified prior to step (e) by alkoxylation such as ethoxylation and/or reaction with monofunctional molecules such as amines, isocyanate, carboxylic acids, alcohols such as mPEG, thiols, esters, acid chlorides, anhydrides, and carbonates.

In one embodiment, the poly-lysine derivative obtained, is modified in step (g) by alkoxylation such as ethoxylation and/or reaction with monofunctional molecules such as amines, isocyanate, carboxylic acids, alcohols such as mPEG, thiols, esters, acid chlorides, anhydrides, and carbonates.

The invention further relates to a non-crosslinked poly-lysine oleate having a weight-average molecular weight in the range of about 20,000 g/mol to about 60,000 g/mol. In one embodiment, said poly-lysine oleate has a polydispersity index (PDI) of about 3.0 to about 10.0. In one embodiment, said poly-lysine oleate is water-soluble.

The invention further relates to a non-crosslinked poly-lysine laurate having a weight-average molecular weight in the range of about 20,000 g/mol to about 85,000 g/mol, or in the range of about 20,000 g/mol to about 60,000 g/mol. In one embodiment, said poly-lysine laurate has a polydispersity index (PDI) of about 3.0 to about 10.0. In one embodiment, said poly-lysine laurate is water-soluble.

The invention also relates to a process for preparation of the poly-lysine derivative of the invention.

The invention provides a liquid composition comprising at least

component A: comprising at least one poly-lysine derivative,

component B: comprising a compound selected from the group of solvents in which component A is soluble, and

optionally component C: at least one additional compound.

The invention provides a process for preparation of the liquid composition of the invention comprising the mixing in no specified order in one or more steps at least component A and component B.

The invention provides a solid-based composition comprising a dispersing medium: comprising at least the liquid composition of the invention comprising at least components A and B, and

component D: at least one solid compound,

wherein the solid compound is insoluble in the dispersing medium of the invention.

The invention also provides a process for preparation of a solid-based composition of the invention comprising the mixing in no specified order in one or more steps component A, component B, optionally component C, and component D. The process for preparation of the solid-based compositions of the invention may include the process of comminution.

The invention provides the use of at least one poly-lysine derivative as wetting and/or dispersing agent for solid particles.

The invention provides the use of at least one poly-lysine derivative in solid-based compositions to increase storage stability of said solid-based composition when compared to solid-based compositions lacking the poly-lysine derivative of the invention.

### DETAILED DESCRIPTION

Lysine, which is the monomer of poly-lysine, is characterized herein by specific positions of consecutive C-atoms starting from the carboxylic group using the Greek alphabet; alpha (α) C is located next to the lysine carboxylic group. It binds a primary amine group. α C is followed by beta (β), gamma (γ), delta (δ) and epsilon (ε) C, the latter binds a primary amine group. In other words, there are amine groups bound in alpha and epsilon position of the lysine molecule. Consequently, lysine molecules during polymerization may result in poly-lysine molecules with branching, when polymerization takes place in alpha and epsilon position.

The current invention relates to a poly-lysine derivative which is obtained by a process comprising the steps of:
(a) heating an aqueous lysine solution to boiling
(b) increasing temperature of the aqueous lysine solution to a reaction temperature in the range of about 105° C. to about 180° C.
(c) keep the reaction temperature in the range of about 105° C. to about 180° C. until
   i. melt viscosity of the reaction mixture in the range of about 350 mPa*s to about 6,500 mPa*s when measured at 160° C. and
   ii. an amine number in the range of about 100 mg KOH/g to about 500 mg KOH/g is achieved
(d) optionally, the vacuum applied is released
(e) add alkyl-carboxylic acid or alkenyl-carboxylic acid in amounts of 2.5 mol % to 10 mol %, relative to the theoretical amount of poly-lysine comprised in the reaction mixture
(f) increase or keep the reaction temperature in the range of about 105° C. to about 180° C. until number of free alkyl-carboxylic acid or alkenyl-carboxylic acid is 9% by weight, relative to the total weight of the reaction mixture,

wherein vacuum is applied either in step (a), (b) and/or (c) and water is removed continuously during the whole process.

Poly-lysine is formed from lysine in a polycondensation reaction in which water is released when an amino group of one lysine molecule and a carboxyl group of another lysine molecule react with each other to form an amide bond. The process according to the invention requires that water is removed. Any means suitable for removing water from the aqueous lysine solution and/or the reaction mixture may be applied. Water may be removed e.g. by adsorption or by distillation.

The term “about,” as used herein, refers to variation in the numerical quantity that can occur, for example, through typical measuring and handling procedures in the real world; through inadvertent error in these procedures; through differences in the manufacture, source, or purity of the ingredients used to make the compositions or carry out the methods; and the like.

The term “reaction temperature” refers to the internal temperature of the reaction mixture in a reaction vessel. The temperature of an external heat source used for heating the reaction vessel may be higher or lower than the reaction temperature.

The aqueous lysine solution and/or the reaction mixture of the invention are part of the “reaction system” which also includes a reaction vessel. The process may be carried out in a continuously or batchwise working reaction system. The process may be carried out in what is called a one-pot mode, in which the lysine is furnished in its entirety in the initial charge and the polycondensation reaction is carried out in a reactor with backmixing. Polycondensation may also be started with only a part of the amount of lysine desired to be furnished in the whole process, wherein the rest of the lysine may be feeded during the polycondensation process batchwise or continuously. Any suitable reaction system may be used such as multistage reactor, a stirred-tank reactor, or a tube reactor. The type of reaction vessel or reactor used, its volume, its isolation measures and other characteristics as well as the actual volume of the reaction mixture in the vessel, have to be recognized during operation accordingly. The one skilled in the art is familiar with the handling of different reactors.

The term “reaction mixture” herein comprises the aqueous lysine solution and/or possible impurities of the same and/or poly-lysine and/or poly-lysine derivative and/or water and/or non-reacted compounds including but not limited to alkenyl-carboxyl acid and/or by-products of the reactions taking place and/or one or more catalysts.

### Step (a):

“Aqueous lysine solution” herein means any aqueous lysine-comprising solution such as fermentation broth comprising lysine. Aqueous lysine solution may also mean that lysine in its solid state has been dissolved in a liquid medium comprising water.

Aqueous lysine solutions of the invention may comprise lysine in amounts of at least 5% by weight, at least 10% by weight, at least 20% by weight, at least 30% by weight, at least 40% by weight, at least 50% by weight, at least 60% by weight, at least 70% by weight, at least 75% by weight, at least 80% by weight, at least 85% by weight, at least 90% by weight, or at least 95% by weight, all relative to the total weight of the aqueous lysine solution. The aqueous lysine solution may comprise L-lysine, D-lysine, or any mixture of L-lysine and D-lysine, e.g. a racemic mixture.

Aqueous lysine solution of the invention comprises water in amounts of about 5% by weight, about 10% by weight, about 15% by weight, about 20%, about 25% by weight, about 30% by weight, about 40% by weight, about 50% by weight, about 60% by weight, about 70% by weight, about 80% by weight, about 90% by weight, or about 95% by weight, all relative to the total weight of the aqueous lysine solution.

Aqueous lysine solution of the invention may comprise impurities such as salts originating from the fermentation medium, cell debris originating from the production host cells, metabolites produced by the production host cells during fermentation.

In one embodiment, impurities are comprised in aqueous lysine solution in amounts less than about 20% by weight, less than about 15% by weight, less than about 10% by weight, or less than about 5% by weight, all relative to the total weight of the aqueous lysine solution.

“Heating to boiling” means increase of the internal temperature of the aqueous lysine solution to at least about 100° C. In one embodiment heating to boiling includes heating to internal temperatures within the aqueous lysine solution in the range of about 100 to about 110° C., or in the range of about 100° C. to about 105° C.

The pressure within the reaction system may be reduced to about 90 kPa, to about 80 kPa, to about 75 kPa, to about 73 kPa, to about 70 kPa, to about 65 kPa, or to about 60 kPa. The reduction of pressure within a reaction system is usually synonymous with “vacuum is applied”. The boiling temperature usually depends on the actual vacuum applied.

In one embodiment, at least one catalyst may be added to the aqueous lysine solution in step (a) in amounts up to 1% by weight relative the total weight of the reaction mixture. As catalyst sodium hypophosphite may be employed in amounts up to 1% by weight relative the total weight of the reaction mixture.

### Step (b):

To start the actual polycondensation reaction, the internal temperature of the reaction mixture is increased to a temperature above boiling temperature, which ranges from about 105° C. to about 180° C. The internal temperature of the reaction mixture may be increased to a temperature in the range of 105° C. to 180° C., in the range of about 135° C. to about 180° C., or in the range of 140° C. to 175° C. In one embodiment, the internal temperature of the reaction mixture is increased to 160° C.

If not done in step (a) already, in one embodiment vacuum is applied in step (b). In one embodiment, pressure within the reaction system has been reduced to a certain extent in step (a) and is further reduced in step (b). The pressure may be reduced as much as for the given reaction system feasible by taking into account that foaming of the reaction mixture has to be avoided. Pressure within the reaction system may be reduced to about 90 kPa, to about 80 kPa, to about 75 kPa, to about 73 kPa, to about 70 kPa, to about 65 kPa, or to about 60 kPa. In one embodiment, vacuum is applied within short time.

In one embodiment, the increase of the internal temperature of the reaction mixture is achieved within short time.

“Within short time” in the context of applying vacuum and/or increase of internal temperature means that the desired pressure reduction and/or increase of the internal temperature of the reaction mixture is achieved within a time-span that is reasonably short for the given reaction system. “Within short time” may mean within 1.5 hours, within 1 hour, within 35 minutes, or within 15 minutes.

In one embodiment, pressure within the reaction system is reduced to about 78 kPa within 35 minutes.

### Step (c):

If not done in step (a) or (b) already, vacuum may be applied in step (c). In one embodiment, pressure within the reaction system has been reduced to a certain extent in step (a) and/or step (b) and is further reduced in step (b). The pressure may be reduced as much as for the given reaction system feasible by taking into account that foaming of the reaction mixture has to be avoided. Pressure within the reaction system may be reduced to about 90 kPa, to about 80 kPa, to about 75 kPa, to about 73 kPa, to about 70 kPa, to about 65 kPa, or to about 60 kPa. In one embodiment, vacuum is applied within short time.

In one embodiment, pressure has already been reduced in step (b) and is further reduced in step (c). For example, pressure may have been reduced within the reaction system in step (b) to 78 kPa within short time and may be further reduced to 73 kPa in step (c) within short time such as 35 minutes.

The desired internal temperature of the reaction mixture once achieved, is kept until
i. melt viscosity of the reaction mixture in the range of about 350 mPa*s to about 6,500 mPa*s is achieved when measured at 160° C., and
ii. an amine number in the range of about 100 mg KOH/g to about 500 mg KOH/g is achieved.

The melt viscosity to be achieved may be in the range of about 350 mPa*s to about 6,500 mPa*s, or in the range of about 1,000 mPa*s to about 6,500 mPa*s when measured at 160° C. The melt viscosity to be achieved may be in the range of 1000 mPa*s to 6,500 mPa*s, in the range of about 3,000 mPa*s to about 6,500 mPa*s, in the range of about 3,500 mPa*s to about 6,500 mPa*s, in the range of about 4,500 mPa*s to about 6,500 mPa*s, or in the range of about 4,500 mPa*s to about 6,200 mPa*s, or in the range of about 5,000 mPa*s to about 6,200 mPa*s, when measured at 140° C.

The melt viscosity values are those determined at 140° C. or 160° C. For the purposes of this invention, the melt viscosity values are determined by melt rheology measurement (plate-plate) using an I.C.I. Cone Plate Viscosimeter from Epprecht GmbH (now Brookfield GmbH). Said melt rheology measurement is to be performed according to DIN 53018.

The amine number to be achieved may be in the range of 100 KOH/g to 500 mg KOH/g, 100 KOH/g to 400 mg KOH/g, in the range of 150 KOH/g to 450 mg KOH/g, in the range of 150 mg KOH/g to 350 mg KOH/g, in the range of 200 KOH/g to 400 mg KOH/g, in the range of 300 KOH/g to 450 mg KOH/g, or in the range of 350 KOH/g to 400 mg KOH/g.

For the purposes of this invention, the amine number is determined by potentiometric titration of the reaction mixture at 20° C. and 101.3 kPa with trifluoromethanesulfonic acid: amount of KOH in mg equals 1 g amine-comprising substance.

In one embodiment, the desired internal temperature of the reaction mixture once achieved, is kept until
i. melt viscosity of the reaction mixture in the range of about 350 mPa*s to about 6,500 mPa*s is achieved, and
ii. an amine number in the range of about 150 mg KOH/g to about 500 mg KOH/g is achieved.

In one embodiment, the reaction mixture is kept at its internal temperature until a K-value of 11-15 or 12-15 is achieved. The reaction mixture may be kept at its internal temperature until a K-value of 11-14, 12-14, 11-13, or 12-13 is achieved.

The K-values are those determined by measurement of kinematic viscosity via Ubbelohde-viscosimeter (DIN 51562-3) at 20° C. and 101.3 kPa.

The end point of the condensation reaction may also be determined via NIR (near infrared) measurement. For this method the amine number which may be determined according to DIN 53176 or the viscosity measurement which may be determined according to DIN 51562-3 is correlated with NIR spectrum followed by subsequent statistical analysis.

The poly-lysine molecule may have a weight-average molecular weight in the range of about 6,000 g/mol to about 30,000 g/mol, in the range of about 6,000 g/mol to about 23,000 g/mol, in the range of about 8,000 g/mol to about 23,000 g/mol, in the range of about 8,000 g/mol to about 20,000 g/mol, in the range of about 8,000 g/mol to about 17,000 g/mol, in the range of about 10,000 g/mol to about 18,000 g/mol, in the range of about 10,000 g/mol to about 17,000 g/mol, or in the range of about 13,000 g/mol to about 17,000 g/mol.

Weight-average molecular weight for the purposes of this invention is to be determined by size exclusion chromatography (SEC or GPC) using hexafluoro iso-propanol with 0.055% of trifluoro acetic acid potassium salt as an eluent at 35° C. Signal detection is performed by UV/Vis and refractive index sensors.

The poly-lysine molecule may have a polydispersity index of ≤5.5, of ≤4.7, of ≤4.5, of ≤4, of ≤3.9, or ≤3.5. The poly-lysine molecule may have a polydispersity index in the range of 2.0 to 4.4, in the range of 2.0 to 4.0, in the range of 2.6 to 3.9, in the range of 2.3 to 3.5, or in the range of 2 to 3.5.

In one embodiment, the poly-lysine molecule has a weight-average molecular weight in the range of about 6,000 g/mol to about 30,000 g/mol and a polydispersity index of ≤5.5, or of ≤4.5.

### Step (d):

Depending on the reaction system used, release of the vacuum applied in steps (a), (b), or (c) may be necessary due to adding further reactants such as alkyl-carboxylic acid or alkenyl-carboxylic acid as described in step (e). Release of vacuum may mean that pressure is increased to about 101.3 kPa.

In one embodiment, the poly-lysine obtained is non-modified poly-lysine which is further processed in step (e).

The melt viscosity of non-modified poly-lysine may be in the range of 500 mPa*s to 3,000 mPa*s, or in the range of about 1,000 mPa*s to about 2,300 mPa*s when measured at 160° C.

The melt viscosity of non-modified poly-lysine may be in the range of 3,000 mPa*s to 6,500 mPa*s, or in the range of about 3,200 mPa*s to about 6,400 mPa*s when measured at 140° C.

In one embodiment, the poly-lysine obtained is modified prior to step (e) by alkoxylation such as ethoxylation (resulting in ethoxylated amine groups) and/or reaction with monofunctional molecules such as amines, isocyanate, carboxylic acids, alcohols such as mPEG, thiols, esters, acid chlorides, anhydrides, and carbonates. The poly-lysine obtained is modified prior to step (e) may be called modified poly-lysine herein.

The melt viscosity of modified poly-lysine, e.g. poly-lysine-mPEG may be in the range of about 350 mPa*s to about 6,500 mPa*s, or in the range of about 350 mPa*s to about 1,000 mPa*s when measured at 160° C. The melt viscosity of modified poly-lysine, e.g. poly-lysine-mPEG may be in the range of about 1,000 mPa*s to about 6,500 mPa*s, or in the range of about 1,000 mPa*s to about 2,000 mPa*s when measured at 140° C.

### Step (e):

Alkyl-carboxylic acid or alkenyl-carboxylic acid is added in amounts in the range of 2.5 mol % to 10 mol %, relative to the theoretical amount of non-modified poly-lysine and/or modified polylysine. The amount of alkyl-carboxylic acid or alkenyl-carboxylic acid added may be in the range of 3 mol % to 8 mol %, or about 5 mol %, all relative to the theoretical amount of non-modified poly-lysine and/or modified poly-lysine comprised in the reaction mixture.

Calculation of molar ratio oleic acid as exemplified for addition of 5 mol % of oleic acid

$$n ( Oleic acid ) = 0.05 * n ( poly \text{-} lysine ) = \frac{m ( poly \text{-} lysine )}{M ( lysine ) - M ( water )}$$

$$molar ratio ( oleic acid ) [ mol \% ] = \frac{n ( Oleic acid )}{n ( poly \text{-} lysine )} * 100$$

The mass of non-modified or modified poly-lysine is calculated from the amount of reaction water to be removed from the reaction mixture. “Reaction water” means the amount of water that evolves from the polymerization reaction.

The addition of alkyl-carboxylic acid or alkenyl-carboxylic acid is to be conducted “within short time”. In any case this relates to avoidance of reduction of the internal temperature of the reaction mixture as far as possible. “Within short time” in the context of adding alkyl-carboxylic acid or alkenyl-carboxylic acid may mean, that the time-span of supplementation should be kept reasonably short for the given reaction system, e.g. by direct feed of the whole volume alkylcarboxylic acid or alkenyl-carboxylic acid into the reaction mixture. “Within short time” in the context of adding alkyl-carboxylic acid or alkenyl-carboxylic acid may also mean, that the time-span during which vacuum is released for the purposes of addition of alkyl-carboxylic acid or alkenyl-carboxylic acid is kept reasonably short for the given reaction system. “Within short time” may mean within about 30 minutes, within about 20 minutes, or within about 10 minutes or less.

Alkyl-carboxylic acid may be C₈-C₂₂ or C₁₂-C₁₈ saturated carboxylic acids.

Alkenyl-carboxylic acid may be selected from C₁₆-C₂₂ mono-, and poly-unsaturated fatty acids. Alkyl-carboxylic acid or alkenyl-carboxylic acid may be oxidized to a certain extent, meaning that this oxidation is naturally occurring by exposure to air. These oxidations may be initiated by e.g. oxygen, ozone and nitrous oxide. Oxidized to a certain extent in this context means, that ≤75% of the oleic acid is oxidized. Oxidized to a certain extent may mean, that ≤70%, ≤65%, ≤60%, ≤55%, ≤50%, ≤45%, or ≤40% oleic acid is oxidized.

In one embodiment, the alkyl-carboxylic acid is lauric acid. Lauric acid for supplementation can be derived from animal or plant origin and constitutes a variety of carbon chain lengths, the predominant being the C₁₂ saturated carboxylic acid. Lauric acid is a major component of coconut oil and palm kernel oil.

Lauric acid may be oxidized to a certain extent. Oxidized to a certain extent in this context means, that ≤75% of the lauric acid is oxidized. Oxidized to a certain extent may mean, that ≤70%, ≤65%, ≤60%, ≤55%, ≤50%, ≤45%, or ≤40% lauric acid is oxidized.

In one embodiment, the alkenyl-carboxylic acid is oleic acid. Oleic acid for supplementation can be derived from animal or plant origin and constitutes a variety of carbon chain lengths, the predominant being the C₁₈ mono- and poly-unsaturated oleic acid. In one embodiment, oleic acid comprises C₁₈ mono-unsaturated oleic acid in amounts of at least 50%. Oleic acid may comprise C₁₈ mono-unsaturated oleic acid in amounts of at least 55%, at least 60%, at least 65%, at least 70%, at least 75%, or at least 80%.

Oleic acid may be oxidized to a certain extent. Oxidized to a certain extent in this context means, that ≤75% of the oleic acid is oxidized. Oxidized to a certain extent may mean, that ≤70%, ≤65%, ≤60%, ≤55%, ≤50%, ≤45%, or ≤40% oleic acid is oxidized.

### Step (f):

If the internal temperature has dropped during addition of alkyl-carboxylic acid or alkenyl-carboxylic acid, the reaction mixture needs again to be increased to the desired internal temperature of the reaction mixture. In one embodiment, this increase is done within short time.

In case, vacuum has been released from the reaction system prior to addition of alkyl-carboxylic acid or alkenyl-carboxylic acid, vacuum may again be applied, meaning that pressure may be reduced to about 90 kPa, to about 80 kPa, to about 75 kPa, to about 73 kPa, to about 70 kPa, to about 65 kPa, or to about 60 kPa. In one embodiment, vacuum is applied within short time. “Within short time” in the context of applying vacuum and/or increase of internal temperature means that the desired pressure reduction and/or increase of the internal temperature of the reaction mixture is achieved within a time-span that is reasonably short for the given reaction system. “Within short time” may mean within ≤1.5 hours, within ≤1 hour, within ≤30 minutes, or within ≤15 minutes.

The desired internal temperature is kept until the number of free alkyl-carboxylic acid or alkenyl-carboxylic acid is ≤9% by weight, relative to the total weight of poly-lysine derivative. The desired internal temperature may be kept until the number of free acid is less ≤8% by weight, ≤5% by weight, ≤2.7% by weight, or ≤2.5% by weight, all relative to the total weight of the reaction mixture.

For the purpose of this invention, free acid is determined by reacting free alkyl-carboxylic acid or alkenyl-carboxylic acid with MSTFA (N-Methyl-N-(trimethylsilyl)trifluoroacetamide) and detecting the resulting alkyl-carboxylic acid or alkenyl-carboxylic acid silyl ester by gaschromatography. The total amount of free alkyl-carboxylic acid or alkenyl-carboxylic acid is determined by adding commercially available standards and by supplementation of alkyl-carboxylic acid or alkenyl-carboxylic acid. The amount of free alkyl-carboxylic acid or alkenyl-carboxylic acid is calculated based on the amount of non-reacted C₁₂-saturated fatty acid or non-reacted C₁₈-mono-unsaturated fatty acid. In one embodiment, free lauric acid is determined by this method, wherein the number of free lauric acid is calculated based on the amount of non-reacted C₁₂-saturated lauric acid. In one embodiment, free oleic acid is determined by this method, wherein the number of free oleic acid is calculated based on the amount of non-reacted C₁₈-mono-unsaturated oleic acid.

In one embodiment, the poly-lysine derivative obtained by the inventive process is a non-modified poly-lysine functionalized with alkyl-carboxylic acid or alkenyl-carboxylic acid which might be called non-modified poly-lysine derivative herein. This is the case if the poly-lysine molecule has not been modified prior to step (e). In one embodiment, the non-modified polylysine is functionalized with oleic acid, which may be called poly-lysine oleate herein. In one embodiment, the non-modified poly-lysine is functionalized with lauric acid, which may be called poly-lysine laurate herein.

In one embodiment, the poly-lysine derivative obtained by the inventive process is a modified poly-lysine functionalized with alkyl-carboxylic acid or alkenyl-carboxylic acid. This is the case if the poly-lysine molecule has been modified prior to step (e). Such a product may be called modified poly-lysine derivative herein. In one embodiment, the modified poly-lysine is functionalized with oleic acid, which may be called modified poly-lysine oleate herein. In one embodiment, the modified poly-lysine is functionalized with lauric acid, which may be called modified poly-lysine laurate herein.

The non-modified poly-lysine derivative and/or the modified poly-lysine derivative obtained by the inventive process may comprise unreacted lysine and/or possible impurities of the same and/or non-modified poly-lysine and/or modified poly-lysine and/or non-modified poly-lysine derivative and/or modified poly-lysine derivative and/or non-reacted compounds and/or by-products of the reactions taking place and/or water and/or one or more catalysts.

The poly-lysine derivative of the invention is non-crosslinked. In one embodiment, the non-modified and/or modified poly-lysine derivative of the invention is non-crosslinked. “Non-crosslinked” means that that there is no deliberate cross-linking in the sense of formation of covalent bounds between single poly-lysine derivative molecules or modified poly-lysine derivative molecules introduced. Therefore, essentially no cross-links are introduced by the process of production as such. Essentially no cross-links may mean that the degree of cross-linking is low, such as below 5%, which might be due to cross-linking substances being present in the reaction mixture as impurities of the aqueous lysine solution, such as arginine.

The poly-lysine derivative obtained by the inventive process may have a weight-average molecular weight in the range of about 20,000 g/mol to about 85,000 g/mol or in the range of about 20,000 g/mol to about 60,000 g/mol. The poly-lysine derivative of the invention may have a weight-average molecular weight in the range of about 30,000 g/mol to about 55,000 g/mol, in the range of about 33,000 g/mol to about 50,000 g/mol, in the range of about 40,000 g/mol to about 55,000 g/mol, or in the range of 40,000 g/mol to 50,000 g/mol. Weight-average molecular weight in this context is to be determined by size exclusion chromatography (SEC or GPC) using hexafluoro iso-propanol as described above.

In one embodiment, the poly-lysine derivative obtained by the inventive process has a polydispersity index in the range of about 3.0 to about 10.0. The poly-lysine derivative of the invention may have a polydispersity index in the range of about 4.0 to about 9.0, in the range of about 4.0 to about 8.0, in the range of about 4.5 to about 7.5, or in the range of about 4.5 to about 7.0.

In one embodiment, the non-modified and/or modified poly-lysine derivative obtained by the inventive process has a polydispersity index in the range of about 3.0 to about 10.0. The non-modified and/or modified poly-lysine derivative of the invention may have a polydispersity index in the range of about 4.0 to about 9.0, in the range of about 4.0 to about 8.0, in the range of about 4.5 to about 7.5, or in the range of about 4.5 to about 7.0.

In one embodiment, the non-modified and/or modified poly-lysine derivative obtained by the inventive process has a K-value of 11-17, of 12-17, of 13-17, of 14-16.5, of 14.5-16.5, or of 15-16.5.

In one embodiment, the poly-lysine derivative obtained by this process is water-soluble. In one embodiment, the non-modified and/or modified poly-lysine derivative obtained by this process is water-soluble.

“Soluble in water” herein means that the non-modified poly-lysine derivative and/or the modified poly-lysine derivative of the invention, is soluble in water till its saturation point is achieved. The saturation point of the non-modified poly-lysine derivative and/or the modified poly-lysine derivative means the point (concentration) where water cannot dissolve any more of the substance at 20° C. and 101.3 kPa. Adding more than this maximum concentration of the non-modified polylysine derivative and/or the modified poly-lysine derivative will result in phase separation (precipitation, flocculation, gelling turbitity).

In one embodiment, the poly-lysine derivative obtained by the inventive process is further processed by the following additional steps:
(g) vacuum applied is released and the temperature within the reaction mixture is reduced to about 150° C. to 100° C.
(h) water is added to yield a solution comprising about 60 parts of poly-lysine derivative and about 40 parts of water.

In one embodiment, the non-modified and/or modified poly-lysine derivative obtained by the inventive process is further processed by the following additional steps:
(g) vacuum applied is released and the temperature within the reaction mixture is reduced to about 150° C. to 100° C.
(h) water is added to yield a poly-lysine derivative solution comprising about 60 parts of modified poly-lysine derivative and about 40 parts of water.

### Step (g):

Release of vacuum usually means that the pressure within the reaction system is increased to atmospheric pressure.

In one embodiment, the non-modified poly-lysine derivative obtained by the inventive process, is modified by alkoxylation such as ethoxylation, resulting in ethoxylated amine groups and/or reaction with monofunctional molecules such as amines, isocyanate, carboxylic acids, alcohols such as mPEG, thiols, esters, acid chlorides, anhydrides, and carbonates.

### Step (h):

The product obtained by the inventive process comprising non-modified and/or modified polylysine derivative is dissolved in water. Said product may be called poly-lysine derivative solution. In one embodiment, the poly-lysine derivative solution comprises non-modified and/or modified poly-lysine derivative.

Dissolving in water may be realized at a temperature of the reaction system of 150° C. to 100° C. Water is preferably added in amounts that viscosity of the product obtained by the inventive process is reduced to an extent that allows handling of the liquid product. The poly-lysine derivative solution may comprise about 60 parts of at least one poly-lysine derivative and about 40 parts water. In one embodiment, the poly-lysine derivative solution comprises about 30 parts of at least one poly-lysine derivative and about 70 parts water. The poly-lysine derivative solution may comprise about 60 parts poly-lysine derivative and about 40 parts water. In one embodiment, the poly-lysine derivative solution comprises about 30 parts of the poly-lysine derivative and about 70 parts water.

In one embodiment, the poly-lysine derivative solution comprises about 60 parts non-modified and/or modified poly-lysine derivative and about 40 parts water. In one embodiment, the polylysine derivative solution comprises about 30 parts non-modified and/or modified poly-lysine derivative and about 70 parts water.

In one embodiment, the pH of the poly-lysine derivative solution is adjusted to a value in the range of 7 to 14 with inorganic or organic acids. The pH of the poly-lysine derivative solution may be adjusted to a value in the range of 7 to 13, in the range of 8-13, in the range of 9-13, or in the range of 9-11 with inorganic or organic bases.

The current invention, in one aspect, relates to a non-crosslinked poly-lysine functionalized with oleic acid. In one embodiment, said non-crosslinked poly-lysine functionalized with oleic acid is water-soluble.

In one embodiment, the non-crosslinked poly-lysine functionalized with oleic acid is a non-crosslinked non-modified poly-lysine functionalized with oleic acid. In one embodiment, the non-crosslinked poly-lysine functionalized with oleic acid is a non-crosslinked modified poly-lysine functionalized with oleic acid.

In one embodiment, the non-crosslinked non-modified poly-lysine functionalized with oleic acid is modified by alkoxylation such as ethoxylation and/or reactions with monofunctional molecules such as amines, isocyanate, carboxylic acids, alcohols such as mPEG, thiols, esters, acid chlorides, anhydrides, and carbonates. In one embodiment, the non-crosslinked modified poly-lysine functionalized with oleic acid is modified by alkoxylation such as ethoxylation and/or reactions with monofunctional molecules such as amines, isocyanate, carboxylic acids, alcohols such as mPEG, thiols, esters, acid chlorides, anhydrides, and carbonates.

The non-crosslinked poly-lysine oleate of the invention has a weight-average molecular weight in the range of about 20,000 g/mol to about 60,000 g/mol. The non-crosslinked poly-lysine oleate of the invention may have a weight-average molecular weight in the range of about 35,000 g/mol to about 55,000 g/mol, in the range of about 35,000 g/mol to about 50,000 g/mol, in the range of about 30,000 g/mol to about 55,000 g/mol, or in the range of 40,000 g/mol to 55,000 g/mol.

The non-crosslinked non-modified poly-lysine oleate of the invention has a weight-average molecular weight in the range of about 20,000 g/mol to about 60,000 g/mol. The non-crosslinked non-modified poly-lysine oleate of the invention may have a weight-average molecular weight in the range of about 35,000 g/mol to about 55,000 g/mol, in the range of about 35,000 g/mol to about 50,000 g/mol, in the range of about 30,000 g/mol to about 55,000 g/mol, or in the range of 40,000 g/mol to 55,000 g/mol.

The non-crosslinked modified poly-lysine oleate of the invention has a weight-average molecular weight in the range of about 20,000 g/mol to about 60,000 g/mol. The non-crosslinked modified poly-lysine oleate of the invention may have a weight-average molecular weight in the range of about 35,000 g/mol to about 55,000 g/mol, in the range of about 35,000 g/mol to about 50,000 g/mol, in the range of about 30,000 g/mol to about 55,000 g/mol, or in the range of 40,000 g/mol to 55,000 g/mol.

Weight-average molecular weight in this context is to be determined by size exclusion chromatography (SEC or GPC) using hexafluoro iso-propanol as described above.

In one embodiment, the non-crosslinked poly-lysine oleate has a polydispersity index in the range of about 3.0 to about 10.0. The non-crosslinked poly-lysine oleate of the invention may have a polydispersity index in the range of about 4.0 to about 8.0, in the range of about 4.6 to about 7.5, or in the range of about 4.6 to about 7.0, or in the range of about 4.5 to about 7.5.

In one embodiment, the non-crosslinked non-modified poly-lysine oleate has a polydispersity index in the range of about 3.0 to about 10.0. The non-crosslinked non-modified poly-lysine oleate of the invention may have a polydispersity index in the range of about 4.0 to about 8.0, in the range of about 4.6 to about 7.5, or in the range of about 4.6 to about 7.0, or in the range of about 4.5 to about 7.5.

In one embodiment, the non-crosslinked modified poly-lysine oleate has a polydispersity index in the range of about 3.0 to about 10.0. The non-crosslinked modified poly-lysine oleate of the invention may have a polydispersity index in the range of about 4.0 to about 8.0, in the range of about 4.6 to about 7.5, or in the range of about 4.6 to about 7.0, or in the range of about 4.5 to about 7.5.

The current invention, in another aspect, relates to a non-crosslinked poly-lysine functionalized with lauric acid. In one embodiment, said non-crosslinked poly-lysine functionalized with lauric acid is water-soluble.

In one embodiment, the non-crosslinked poly-lysine functionalized with lauric acid is a non-crosslinked non-modified poly-lysine functionalized with lauric acid. In one embodiment, the non-crosslinked poly-lysine functionalized with lauric acid is a non-crosslinked modified polylysine functionalized with lauric acid.

In one embodiment, the non-crosslinked non-modified poly-lysine functionalized with lauric acid is modified by alkoxylation such as ethoxylation and/or reactions with monofunctional molecules such as amines, isocyanate, carboxylic acids, alcohols such as mPEG, thiols, esters, acid chlorides, anhydrides, and carbonates. In one embodiment, the non-crosslinked modified poly-lysine functionalized with lauric acid is modified by alkoxylation such as ethoxylation and/or reactions with monofunctional molecules such as amines, isocyanate, carboxylic acids, alcohols such as mPEG, thiols, esters, acid chlorides, anhydrides, and carbonates.

The non-crosslinked poly-lysine laurate of the invention has a weight-average molecular weight in the range of about 20,000 g/mol to about 60,000 g/mol. The non-crosslinked poly-lysine laurate of the invention may have a weight-average molecular weight in the range of about 30,000 g/mol to about 55,000 g/mol, or in the range of 40,000 g/mol to 55,000 g/mol.

The non-crosslinked non-modified poly-lysine laurate of the invention may have a weight-average molecular weight in the range of about 20,000 g/mol to about 85,000 g/mol, in the range of about 20,000 g/mol to about 82,000 g/mol, or in the range of about 20,000 g/mol to about 60,000 g/mol. The non-crosslinked non-modified poly-lysine laurate of the invention may have a weight-average molecular weight in the range of about 30,000 g/mol to about 82,000 g/mol, in the range of about 30,000 g/mol to about 55,000 g/mol, in the range of about 40,000 g/mol to about 82,000 g/mol, or in the range of 40,000 g/mol to 55,000 g/mol.

The non-crosslinked modified poly-lysine laurate of the invention has a weight-average molecular weight in the range of about 20,000 g/mol to about 85,000 g/mol, in the range of about 20,000 g/mol to about 82,000 g/mol, or in the range of about 20,000 g/mol to about 60,000 g/mol. The non-crosslinked modified poly-lysine laurate of the invention may have a weight-average molecular weight in the range of about 30,000 g/mol to about 82,000 g/mol, in the range of about 30,000 g/mol to about 55,000 g/mol, in the range of about 40,000 g/mol to about 82,000 g/mol, or in the range of 40,000 g/mol to 55,000 g/mol.

Weight-average molecular weight in this context is to be determined by size exclusion chromatography (SEC or GPC) using hexafluoro iso-propanol as described above.

In one embodiment, the non-crosslinked poly-lysine laurate has a polydispersity index in the range of about 3.0 to about 10.0. The non-crosslinked poly-lysine laurate of the invention may have a polydispersity index in the range of about 4.0 to about 9.0, in the range of about 4.0 to about 8.0, in the range of about 4.5 to about 7.5, or in the range of about 8.0 to about 9.0.

In one embodiment, the non-crosslinked non-modified poly-lysine laurate has a polydispersity index in the range of about 3.0 to about 10.0. The non-crosslinked non-modified poly-lysine laurate of the invention may have a polydispersity index in the range of about 4.0 to about 9.0, in the range of about 4.0 to about 8.0, in the range of about 4.5 to about 7.5, or in the range of about 8.0 to about 9.0.

In one embodiment, the non-crosslinked modified poly-lysine laurate has a polydispersity index in the range of about 3.0 to about 10.0. The non-crosslinked modified poly-lysine laurate of the invention may have a polydispersity index in the range of about 4.0 to about 9.0, in the range of about 4.0 to about 8.0, in the range of about 4.5 to about 7.5, or in the range of about 8.0 to about 9.0.

The current invention relates to a process for preparation of poly-lysine derivative, wherein the process comprises the steps of:
(a) heating an aqueous lysine solution to boiling
(b) increasing temperature of the aqueous lysine solution to a reaction temperature in the range of about 105° C. to about 180° C.
(c) keep the reaction temperature in the range of about 105° C. to about 180° C. until
   i. melt viscosity of the reaction mixture in the range of about 350 mPa*s to about 6,500 mPa*s and
   ii. an amine number in the range of about 100 mg KOH/g to about 500 mg KOH/g is achieved
(d) optionally, the vacuum applied is released
(e) add alkyl-carboxylic acid or alkenyl-carboxylic acid in amounts of 2.5 mol % to 10 mol %, relative to the theoretical amount of poly-lysine comprised in the reaction mixture
(f) increase or keep the reaction temperature in the range of about 105° C. to about 180° C. until number of free alkyl-carboxylic acid or alkenyl-carboxylic acid is 59% by weight, relative to the total weight of the reaction mixture,

wherein vacuum is applied either in step (a), (b) and/or (c) and water is removed continuously during the whole process.

In one embodiment, the process for preparation of poly-lysine derivative, comprises the following additional steps:
(g) vacuum applied is released and the temperature within the reaction mixture is reduced to about 150° C. to 100° C.
(h) water is added to yield a poly-lysine derivative solution comprising about 60 parts of at least one poly-lysine derivative and about 40 parts water.

In one embodiment, the process for preparation of poly-lysine derivative, comprises the following additional steps:
(g) vacuum applied is released and the temperature within the reaction mixture is reduced to about 150° C. to 100° C.
(h) water is added to yield a poly-lysine derivative solution comprising about 60 parts polylysine derivative and about 40 parts water.

In one embodiment, the process for preparation of poly-lysine derivative, comprises the following additional steps:
(g) vacuum applied is released and the temperature within the reaction mixture is reduced to about 150° C. to 100° C.
(h) water is added to yield a poly-lysine derivative solution comprising about 60 parts non-modified and/or modified poly-lysine derivative and about 40 parts water.

The steps, specifics, and embodiments of the process are those as disclosed above.

The invention relates to a liquid composition comprising at least

component A: comprising at least one poly-lysine derivative of the invention and

component B: comprising a compound selected from the group of solvents in which component A is soluble and

optionally component C: comprising at least one additional compound.

The inventive compositions are liquid at 20° C. and 101.3 kPa. Liquid in this context includes any pourable liquids. In the context of the present invention, gel-type compositions are a special embodiment of liquid compositions. Gel-type compositions may comprise at least one rheology modifier, and they may comprise little or no non-aqueous solvents. Gel-type compositions may contain at least one rheology modifier, and they may contain little or no non-aqueous solvents. In one embodiment, component A comprises at least one non-modified and/or modified polylysine derivative. In one embodiment, component A comprises at least one non-modified polylysine derivative which has been modified by alkoxylation such as ethoxylation and/or reaction with monofunctional molecules such as amines, isocyanate, carboxylic acids, alcohols such as mPEG, thiols, esters, acid chlorides, anhydrides, and carbonates.

In one embodiment, component A comprises at least one non-modified and/or modified polylysine oleate. In one embodiment, component A comprises at least one non-modified poly-lysine oleate which has been modified by alkoxylation such as ethoxylation and/or reaction with monofunctional molecules such as amines, isocyanate, carboxylic acids, alcohols such as mPEG, thiols, esters, acid chlorides, anhydrides, and carbonates.

In one embodiment, component A comprises at least one non-modified and/or modified polylysine laurate. In one embodiment, component A comprises at least one non-modified polylysine laurate which has been modified by alkoxylation such as ethoxylation and/or reaction with monofunctional molecules such as amines, isocyanate, carboxylic acids, alcohols such as mPEG, thiols, esters, acid chlorides, anhydrides, and carbonates.

In one embodiment, component A comprises at least two poly-lysine derivatives selected from the group of non-modified poly-lysine derivative, modified poly-lysine derivative, and non-modified poly-lysine derivative which has been modified by alkoxylation such as ethoxylation and/or reaction with monofunctional molecules such as amines, isocyanate, carboxylic acids, alcohols such as mPEG, thiols, esters, acid chlorides, anhydrides, and carbonates.

Component B comprises at least one compound selected from the group of solvents in which component A is soluble. In one embodiment, component A is soluble in at least one solvent comprised in component B at 20° C. and 101.3 kPa to form a homogenous solution.

“Soluble” in solvent herein means that the poly-lysine derivative is soluble in the solvent till the saturation point of the poly-lysine derivative is achieved. The saturation point of the poly-lysine derivative is usually the point (concentration) where at least one solvent cannot dissolve any more of the poly-lysine derivative at 20° C. and 101.3 kPa. Adding more than this maximum concentration of the substance will result in phase separation (precipitation, flocculation, gelling turbitity).

Suitable solvents are water, organic solvents such as mineral oil fractions of medium to high boiling point, coal tar oils and oils of vegetable or animal origin, aliphatic, cyclic and aromatic hydrocarbons (e.g. paraffins, tetrahydronaphthalene, alkylated naphthalenes and their derivatives, alkylated benzenes and their derivatives), alcohols, glycols, ketones, fatty acid dimethylamides, fatty acids and fatty acid esters and strongly polar solvents.

In one embodiment, the solvents comprised in component B are miscible with each other. Miscible with each other means, that no phase separation takes place between the solvents mixed. In one embodiment, at least one non-modified and/or poly-lysine derivative comprised in component A is soluble in at least one solvent comprised in component B to form a homogenous solution at 20° C. and 101.3 kPa. In one embodiment, at least one non-modified poly-lysine derivative which has been modified by alkoxylation such as ethoxylation and/or reaction with monofunctional molecules such as amines, isocyanate, carboxylic acids, alcohols such as mPEG, thiols, esters, acid chlorides, anhydrides, and carbonates. comprised in component A is soluble in at least one solvent comprised in component B to form a homogenous solution at 20° C. and 101.3 kPa.

In one embodiment, component B comprises a mixture of two or more solvents, wherein at least one poly-lysine derivative comprised in component A is soluble in the mixture of the two or more solvents to form a homogenous solution at 20° C. and 101.3 kPa.

In one embodiment, at least one non-modified and/or modified poly-lysine derivative comprised in component A is soluble in a mixture of two or more solvents comprised in component B to form a homogenous solution at 20° C. and 101.3 kPa. In one embodiment, at least one non-modified poly-lysine derivative which has been modified by alkoxylation such as ethoxylation and/or reaction with monofunctional molecules such as amines, isocyanate, carboxylic acids, alcohols such as mPEG, thiols, esters, acid chlorides, anhydrides, and carbonates comprised in component A is soluble in a mixture of two or more solvents comprised in component B to form a homogenous solution at 20° C. and 101.3 kPa.

In one embodiment, at least one solvent is water-miscible. Water-miscible solvents include aprotic polar solvents and protic solvents. Non-limiting examples of aprotic polar solvents include ketones (e.g. cyclohexanone), lactones (e.g. gamma-butyrolactone), lactames (e.g. N-methyl-2-pyrrolidone), nitriles, tertiary carbonic acid amides, sulfoxides, and carbonates. Non-limiting examples of protic solvents include aliphatic alcohols (e.g. ethanol, propanol, butanol, benzyl alcohol and cyclohexanol), glycols, primary and secondary carbonic acid amides.

Liquid compositions of the invention may have a total solvent content in the range of about 10% to about 90% by weight, in the range of about 10% to about 80% by weight, of about 20% to about 80% by weight, of about 20% to about 70% by weight, of about 30% to about 70% by weight, or of about 40% to about 70% by weight, all relative to the total weight of the liquid composition.

In one embodiment, at least one solvent is water. In one embodiment, component B comprises water and at least one additional solvent which is miscible with water.

In one embodiment, liquid compositions of the present invention have a water content in the range of about 10% to about 90% by weight relative to the total weight of the liquid composition. Liquid compositions of the present invention may have a water content in the range of about 10% to about 80% by weight, of about 20% to about 80% by weight, of about 20% to about 70% by weight, of about 30% to about 70% by weight, or of about 40% to about 70% by weight, all relative to the total weight of the liquid composition.

In one embodiment, at least one additional compound comprised in component C is selected from the group of preservatives.

Preservatives are usually added to liquid compositions to prevent alterations of said compositions due to attacks from microorganisms. Non-limiting examples of suitable preservatives include (quaternary) ammonium compounds, isothiazolinones, organic acids, and formaldehyde releasing agents. Non-limiting examples of suitable (quaternary) ammonium compounds include benzalkonium chlorides, polyhexamethylene biguanide (PHMB), Didecyldimethylammonium chloride(DDAC), and N-(3-aminopropyl)-N-dodecylpropane-1,3-diamine (Diamine). Non-limiting examples of suitable isothiazolinones include 1,2-benzisothiazolin-3-one (BIT), 2-methyl-2H-isothiazol-3-one (MIT), 5-chloro-2-methyl-2H-isothiazol-3-one (CIT), 2-octyl-2H-isothiazol-3-one (OIT), and 2-butyl-benzo[d]isothiazol-3-one (BBIT). Non-limiting examples of suitable organic acids include benzoic acid, sorbic acid, L-(+)-lactic acid, formic acid, and salicylic acid. Non-limiting examples of suitable formaldehyde releasing agent include N,N′-methylenebismorpholine (MBM), 2,2′,2″-(hexahydro-1,3,5-triazine-1,3,5-triyl)triethanol (HHT), (ethylenedioxy)dimethanol, .alpha.,.alpha.′,.alpha.″-trimethyl-1,3,5-triazine-1,3,5(2H,4H,6H)triethanol (HPT), 3,3′-methylenebis[5-methyloxazolidine] (MBO), and cis-1-(3-chloroallyl)-3,5,7-triaza-1-azoniaadamantane chloride (CTAC).

Further useful preservatives include iodopropynyl butylcarbamate (IPBC), halogen releasing compounds such as dichloro-dimethyl-hydantoine (DCDMH), bromo-chloro-dimethyl-hydantoine (BCDMH), and dibromo-dimethyl-hydantoine (DBDMH); bromo-nitro compounds such as Bronopol (2-bromo-2-nitropropane-1,3-diol), 2,2-dibromo-2-cyanoacetamide (DBNPA); aldehydes such as glutaraldehyde; phenoxyethanol; Biphenyl-2-ol; and zinc or sodium pyrithione.

In one embodiment, at least one additional compound comprised in component C is selected from the group of surfactants.

“Surfactant” (synonymously used herein with “surface active agent”) herein means any organic chemical that, when added to a liquid, changes the properties of that liquid at an interface. According to its ionic charge, a surfactant is called non-ionic, anionic, cationic, or amphoteric.

In one embodiment, component C comprises at least one non-ionic surfactant. Non-ionic surfactant herein means a surfactant that contains neither positively nor negatively charged (i.e. ionic) functional groups. Examples provided below for surfactants of any kind are to be understood to be non-limiting.

Non-ionic surfactants may be compounds of the general formulae (Ia) and (Ib):

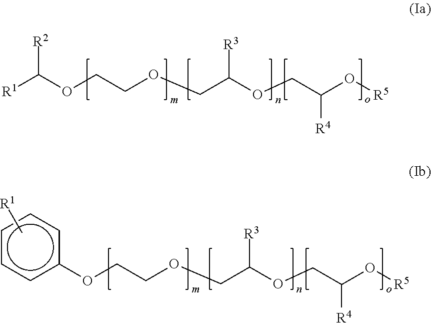

The variables of the general formulae (Ia) and (Ib) are defined as follows:

R¹ is selected from C₁-C₂₃ alkyl and C₂-C₂₃ alkenyl, wherein alkyl and/or alkenyl are linear or branched; examples are n-C₇H₁₅, n-C₉H₁₉, n-C₁₁H₂₃, n-C₁₃H₂₇, n-C₁₅H₃₁, n-C₁₇H₃₅, i-C₉H₁₉, i-C₁₂H₂₅.

R² is selected from H, C₁-C₂₀ alkyl and C₂-C₂₀ alkenyl, wherein alkyl and/or alkenyl are linear or branched.

R³ and R⁴, each independently selected from C₁-C₁₆ alkyl, wherein alkyl is linear or branched; examples are methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl, isodecyl.

R⁵ is selected from H and C₁-C₁₈ alkyl, wherein alkyl is linear or branched.

The integers of the general formulae (la) and (Ib) are defined as follows:

m is in the range of zero to 200, preferably 1-80, more preferably 3-20; n and o, each independently in the range of zero to 100; n preferably is in the range of 1 to 10, more preferably 1 to 6; o preferably is in the range of 1 to 50, more preferably 4 to 25. The sum of m, n and o is at least one, preferably the sum of m, n and o is in the range of 5 to 100, more preferably in the range of from 9 to 50.

The non-ionic surfactants of the general formula (I) may be of any structure, is it block or random structure, and is not limited to the displayed sequence of formula (I).

Non-ionic surfactants may further be compounds of the general formula (II), which might be called alkyl-polyglycosides (APG):

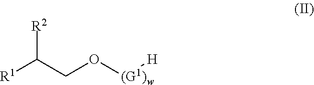

The variables of the general formula (II) are defined as follows:

R¹ is selected from C₁-C₁₇ alkyl and C₂-C₁₇ alkenyl, wherein alkyl and/or alkenyl are linear or branched; examples are n-C₇H₁₅, n-C₉H₁₉, n-C₁₁H₂₃, n-C₁₃H₂₇, n-C₁₅H₃₁, n-C₁₇H₃₅, i-C₉H₁₉, i-C₁₂H₂₅.

R² is selected from H, C₁-C₁₇ alkyl and C₂-C₁₇ alkenyl, wherein alkyl and/or alkenyl are linear or branched.

G¹ is selected from residues of monosaccharides with 4 to 6 carbon atoms, such as glucose and xylose.

The integer w of the general formula (II) is in the range of from 1.1 to 4, w being an average number.

Non-ionic surfactants may further be compounds of general formula (III):

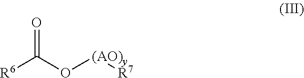

The variables of the general formula (III) are defined as follows:

AO is selected from ethylene oxide (EO), propylene oxide (PO), butylene oxide (BO), and mixtures thereof.

R⁶ is selected from C₅-C₁₇ alkyl and C₅-C₁₇ alkenyl, wherein alkyl and/or alkenyl are linear or branched.

R⁷ is selected from H, C₁-C₁₈-alkyl, wherein alkyl is linear or branched.

The integer y of the general formula (III) is a number in the range of 1 to 70, preferably 7 to 15.

Non-ionic surfactants may further be selected from sorbitan esters and/or ethoxylated or propoxylated sorbitan esters. Non-limiting examples are products sold under the trade names SPAN and TWEEN.

Non-ionic surfactants may further be selected from alkoxylated mono- or di-alkylamines, fatty acid monoethanolamides (FAMA), fatty acid diethanolamides (FADA), ethoxylated fatty acid monoethanolamides (EFAM), propoxylated fatty acid monoethanolamides (PFAM), polyhydroxy alkyl fatty acid amides, or N-acyl N-alkyl derivatives of glucosamine (glucamides, GA, or fatty acid glucamide, FAGA), and combinations thereof.

In one embodiment of the invention, component C comprises two or more different non-ionic surfactants.

In one embodiment, component C comprises at least one amphoteric surfactant. Amphoteric surfactants are those, depending on pH, which can be either cationic, zwitterionic or anionic. Amphoteric surfactants may be compounds comprising amphoteric structures of general formula (IV), which might be called modified amino acids (proteinogenic as well as nonproteinogenic):

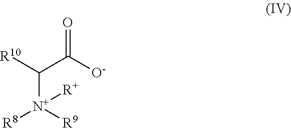

The variables in general formula (IV) are defined as follows:

R⁸ is selected from H, C₁-C₄ alkyl, C₂-C₄ alkenyl, wherein alkyl and/or are linear or branched.

R⁹ is selected from C₁-C₂₂-alkyl, C₂-C₂₂-alkenyl, C₁₀-C₂₂ alkylcarbonyl, and C₁₀-C₂₂ alkenylcarbonyl.

R¹⁰ is selected from H, methyl, —(CH₂)₃NHC(NH)NH₂, —CH₂C(O)NH₂, —CH₂C(O)OH, —(CH₂)₂C(O)NH₂, —(CH₂)₂C(O)OH, (imidazole-4-yl)-methyl, —CH(CH₃)C₂H₅, —CH₂CH(CH₃)₂, —(CH₂)₄NH₂, benzyl, hydroxymethyl, —CH(OH)CH₃, (indole-3-yl)-methyl, (4-hydroxy-phenyl)methyl, isopropyl, —(CH₂)₂SCH₃, and —CH₂SH.

R^{x} is selected from H and C₁-C₄-alkyl.

Amphoteric surfactants may further be compounds comprising amphoteric structures of general formulae (Va), (Vb), or (Vc), which might be called betaines and/or sulfobetaines:

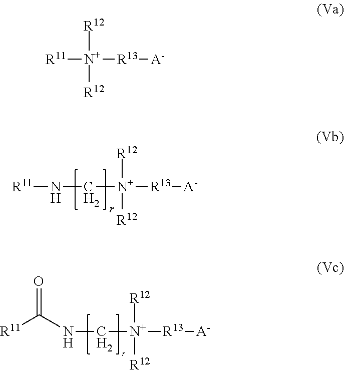

The variables in general formulae (Va), (Vb) and (Vc) are defined as follows:

R¹¹ is selected from linear or branched C₇-C₂₂ alkyl and linear or branched C₇-C₂₂ alkenyl.

R¹² are each independently selected from linear C₁-C₄ alkyl.

R¹³ is selected from C₁-C₅ alkyl and hydroxy C₁-C₅ alkyl; for example 2-hydroxypropyl.

A⁻ is selected from carboxylate and sulfonate.

The integer r in general formulae (Va), (Vb), and (Vc) is in the range of 2 to 6.

Amphoteric surfactants may further be compounds comprising amphoteric structures of general formula (VI), which might be called alkyl-amphocarboxylates:

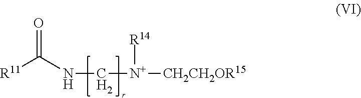

The variables in general formula (VI) are defined as follows:

R¹¹ is selected from C₇-C₂₂ alkyl and C₇-C₂₂ alkenyl, wherein alkyl and/or alkenyl are linear or branched, preferably linear.

R¹⁴ is selected from —CH₂C(O)O⁻M⁺, —CH₂CH₂C(O)O⁻M⁺ and —CH₂CH(OH)CH₂SO₃⁻M⁺.

R¹⁵ is selected from H and —CH₂C(O)O⁻

The integer r in general formula (VI) is in the range of 2 to 6.

Non-limiting examples of further suitable alkyl-amphocarboxylates include sodium cocoamphoacetate, sodium lauroamphoacetate, sodium capryloamphoacetate, disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, and disodium capryloamphodipropionate.

Amphoteric surfactants may further be compounds comprising amphoteric structures of general formula (VII), which might be called amine oxides (AO):

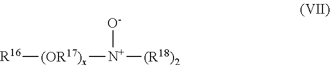

The variables in general formula (VII) are defined as follows:

R¹⁶ is selected from C₈-C₁₈ linear or branched alkyl, hydroxy C₈-C₁₈ alkyl, acylamidopropoyl and C₈-C₁₈ alkyl phenyl group; wherein alkyl and/or alkenyl are linear or branched.

R¹⁷ is selected from C₂-C₃ alkylene, hydroxy C₂-C₃ alkylene, and mixtures thereof.

R¹⁸: each residue can be independently selected from C₁-C₃ alkyl and hydroxy C₁-C₃; R¹⁵ groups can be attached to each other, e.g., through an oxygen or nitrogen atom, to form a ring structure.

The integer x in general formula (VII) is in the range of 0 to 5, preferably from 0 to 3, most preferably 0.

Non-limiting examples of further suitable amine oxides include C₁₀-C₁₈ alkyl dimethyl amine oxides and C₈-C₁₈ alkoxy ethyl dihydroxyethyl amine oxides. Examples of such materials include dimethyloctyl amine oxide, diethyldecyl amine oxide, bis-(2-hydroxyethyl)dodecyl amine oxide, dimethyldodecylamine oxide, dipropyltetradecyl amine oxide, methylethylhexadecyl amine oxide, dodecylamidopropyl dimethyl amine oxide, cetyl dimethyl amine oxide, stearyl dimethyl amine oxide, tallow dimethyl amine oxide and dimethyl-2-hydroxyoctadecyl amine oxide.

A further example of a suitable amine oxide is cocamidylpropyl dimethylaminoxide, sometimes also called cocamidopropylamine oxide.

In one embodiment, component C comprises two or more different amphoteric surfactants.

In one embodiment, component C comprises at least one anionic surfactant. Anionic surfactant means a surfactant with a negatively charged ionic group. Anionic surfactants include, but are not limited to, surface-active compounds that contain a hydrophobic group and at least one water-solubilizing anionic group, usually selected from sulfates, sulfonate, and carboxylates to form a water-soluble compound.

Anionic surfactants may be compounds of general formula (VIII), which might be called (fatty) alcohol/alkyl (ethoxy/ether) sulfates [(F)A(E)S] when A⁻ is SO₃⁻, (fatty) alcohol/alkyl (ethoxy/ether) carboxylat [(F)A(E)C] when A⁻ is —RCOO⁻:

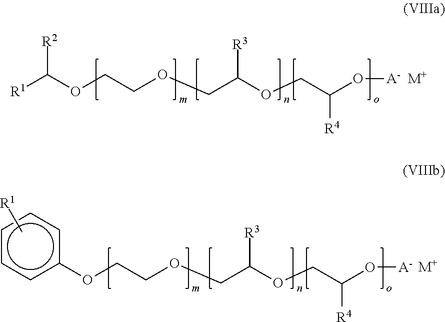

The variables in general formulae (VIIIa and VIIIb) are defined as follows:

R¹ is selected from C₁-C₂₃-alkyl (such as 1-, 2-, 3-, 4-C₁-C₂₃-alkyl) and C₂-C₂₃-alkenyl, wherein alkyl and/or alkenyl are linear or branched, and wherein 2-, 3-, or 4-alkyl; examples are n-C₇H₁₅, n-C₉H₁₉, n-C₁₁H₂₃, n-C₁₃H₂₇, n-C₁₅H₃₁, n-C₁₇H₃₅, i-C₉H₁₉, i-C₁₂H₂₅.

R² is selected from H, C₁-C₂₀-alkyl and C₂-C₂₀-alkenyl, wherein alkyl and/or alkenyl are linear or branched.

R³ and R⁴, each independently selected from C₁-C₁₆-alkyl, wherein alkyl is linear or branched; examples are methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl, isodecyl.

A⁻ is selected from —RCOO⁻, —SO₃⁻ and RSO₃⁻, wherein R is selected from linear or branched C₁-C₈-alkyl, and C₁-C₄ hydroxyalkyl, wherein alkyl is.

M⁺ is selected from H and salt forming cations. Salt forming cations may be monovalent or multivalent; hence M⁺ equals 1/v M^{v+}. Examples include but are not limited to sodium, potassium, magnesium, calcium, ammonium, and the ammonium salt of mono-, di, and triethanolamine.

The integers of the general formulae (VIIIa) and (VIIIb) are defined as follows:

m is in the range of zero to 200, preferably 1-80, more preferably 3-20; n and o, each independently in the range of zero to 100; n preferably is in the range of 1 to 10, more preferably 1 to 6; o preferably is in the range of 1 to 50, more preferably 4 to 25. The sum of m, n and o is at least one, preferably the sum of m, n and o is in the range of 5 to 100, more preferably in the range of from 9 to 50.

Anionic surfactants of the general formula (VIII) may be of any structure, block copolymers or random copolymers.

Further suitable anionic surfactants include salts (M⁺) of C₁₂-C₁₈ sulfo fatty acid alkyl esters (such as C₁₂-C₁₈ sulfo fatty acid methyl esters), C₁₀-C₁₈-alkylarylsulfonic acids (such as n-C₁₀-C₁₈-alkylbenzene sulfonic acids) and C₁₀-C₁₈ alkyl alkoxy carboxylates.

M⁺ in all cases is selected from salt forming cations. Salt forming cations may be monovalent or multivalent; hence M⁺ equals 1/v M^{v+}. Examples include but are not limited to sodium, potassium, magnesium, calcium, ammonium, and the ammonium salt of mono-, di, and triethanolamine.

Non-limiting examples of further suitable anionic surfactants include branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, secondary alkanesulfonates (SAS), paraffin sulfonates (PS), sulfonated fatty acid glycerol esters, alkyl- or alkenylsuccinic acid, fatty acid derivatives of amino acids, diesters and monoesters of sulfo-succinic acid.

Anionic surfactants may be compounds of general formula (IX), which might be called N-acyl amino acid surfactants:

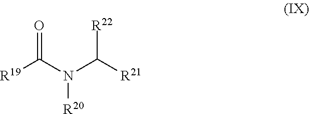

The variables in general formula (IX) are defined as follows:

R¹⁹ is selected from linear or branched C₆-C₂₂-alkyl and linear or branched C₆-C₂₂-alkenyl such as oleyl.

R²⁰ is selected from H and C₁-C₄-alkyl.

R²¹ is selected from H, methyl, —(CH₂)₃NHC(NH)NH₂, —CH₂C(O)NH₂, —CH₂C(O)OH, —(CH₂)₂C(O)NH₂, —(CH₂)₂C(O)OH, (imidazole-4-yl)-methyl, —CH(CH₃)C₂H₅, —CH₂CH(CH₃)₂, —(CH₂)₄NH₂, benzyl, hydroxymethyl, —CH(OH)CH₃, (indole-3-yl)-methyl, (4-hydroxy-phenyl)methyl, isopropyl, —(CH₂)₂SCH₃, and —CH₂SH.

R²² is selected from —COOX and —CH₂SO₃X, wherein X is selected from Li⁺, Na⁺ and K⁺.

Non-limiting examples of suitable N-acyl amino acid surfactants are the mono- and dicarboxylate salts (e.g., sodium, potassium, ammonium and ammonium salt of mono-, di, and triethanolamine) of N-acylated glutamic acid, for example, sodium cocoyl glutamate, sodium lauroyl glutamate, sodium myristoyl glutamate, sodium palmitoyl glutamate, sodium stearoyl glutamate, disodium cocoyl glutamate, disodium stearoyl glutamate, potassium cocoyl glutamate, potassium lauroyl glutamate, and potassium myristoyl glutamate; the carboxylate salts (e.g., sodium, potassium, ammonium and ammonium salt of mono-, di, and triethanolamine) of N-acylated alanine, for example, sodium cocoyl alaninate, and triethanolamine lauroyl alaninate; the carboxylate salts (e.g., sodium, potassium, ammonium and ammonium salt of mono-, di, and triethanolamine) of N-acylated glycine, for example, sodium cocoyl glycinate, and potassium cocoyl glycinate; the carboxylate salts (e.g., sodium, potassium, ammonium and ammonium salt of mono-, di, and triethanolamine) of N-acylated sarcosine, for example, sodium lauroyl sarcosinate, sodium cocoyl sarcosinate, sodium myristoyl sarcosinate, sodium oleoyl sarcosinate, and ammonium lauroyl sarcosinate.

Anionic surfactants may further be selected from the group of soaps. Suitable are salts (M⁺) of saturated and unsaturated C₁₂-C₁₈ fatty acids, such as lauric acid, myristic acid, palmitic acid, stearic acid, behenic acid, oleic acid, (hydrated) erucic acid. M⁺ is selected from salt forming cations. Salt forming cations may be monovalent or multivalent; hence M⁺ equals 1/v M^{v+}. Examples include but are not limited to sodium, potassium, magnesium, calcium, ammonium, and the ammonium salt of mono-, di, and triethanolamine.

Further non-limiting examples of suitable soaps include soap mixtures derived from natural fatty acids such as tallow, coconut oil, palm kernel oil, laurel oil, olive oil, or canola oil. Such soap mixtures comprise soaps of lauric acid and/or myristic acid and/or palmitic acid and/or stearic acid and/or oleic acid and/or linoleic acid in different amounts, depending on the natural fatty acids from which the soaps are derived.

Further non-limiting examples of suitable anionic surfactants include salts (M⁺) of sulfates, sulfonates or carboxylates derived from natural fatty acids such as tallow, coconut oil, palm kernel oil, laurel oil, olive oil, or canola oil. Such anionic surfactants comprise sulfates, sulfonates or carboxylates of lauric acid and/or myristic acid and/or palmitic acid and/or stearic acid and/or oleic acid and/or linoleic acid in different amounts, depending on the natural fatty acids from which the soaps are derived.

In one embodiment, component C comprises two or more different anionic surfactants.

In one embodiment, component C comprises at least one cationic surfactant. Cationic surfactant means a surfactant with a positively charged ionic group. Typically, these cationic moieties are nitrogen containing groups such as quaternary ammonium or protonated amino groups. The cationic protonated amines can be primary, secondary, or tertiary amines.

Cationic surfactants may be compounds of the general formula (X) which might be called quaternary ammonium compounds (quats):

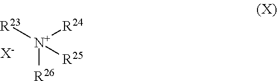

The variables in general formula (X) are defined as follows:

R²³ is selected from H, C₁-C₄ alkyl (such as methyl) and C₂-C₄ alkenyl, wherein alkyl and/or alkenyl is linear or branched.

R²⁴ is selected from C₁-C₄ alkyl (such as methyl), C₂-C₄ alkenyl and C₁-C₄ hydroxyalkyl (such as hydroxyethyl), wherein alkyl and/or alkenyl is linear or branched.

R²⁵ is selected from C₁-C₂₂ alkyl (such as methyl, C₁₈ alkyl), C₂-C₄ alkenyl, C₁₂-C₂₂ alkylcarbonyloxymethyl and C₁₂-C₂₂ alkylcarbonyloxyethyl (such as C₁₆-C₁₈ alkylcarbonyloxyethyl), wherein alkyl and/or alkenyl is linear or branched.

R²⁶ is selected from C₁₂-C₁₈ alkyl, C₂-C₄ alkenyl, C₁₂-C₂₂ alkylcarbonyloxymethyl, C₁₂-C₂₂ alkylcarbonyloxyethyl and 3-(C₁₂-C₂₂ alkylcarbonyloxy)-2(C₁₂-C₂₂ alkylcarbonyloxy)-propyl.

X⁻ is selected from halogenid, such as Cl⁻ or Br⁻.

Non-limiting examples of further cationic surfactants include, amines such as primary, secondary and tertiary monoamines with C₁₈ alkyl or alkenyl chains, ethoxylated alkylamines, alkoxylates of ethylenediamine, imidazoles (such as 1-(2-hydroxyethyl)-2-imidazoline, 2-alkyl-1-(2-hydroxyethyl)-2-imidazoline, and the like), quaternary ammonium salts like alkylquaternary ammonium chloride surfactants such as n-alkyl(C₁₂-C₁₈)dimethylbenzyl ammonium chloride, ntetradecyldimethylbenzylammonium chloride monohydrate, and a naphthylene-substituted quaternary ammonium chloride such as dimethyl-1-naphthylmethylammonium chloride.

Particularly suitable cationic surfactants that may be:
N,N-dimethyl-N-(hydroxy-C₇-C₂₅-alkyl)ammonium salts;
mono- and di(C₇-C₂₅-alkyl)dimethylammonium compounds quaternized with alkylating agents;
ester quats, in particular quaternary esterified mono-, di- and trialkanolamines which are esterified with C₈-C₂₂-carboxylic acids;
imidazoline quats, in particular 1-alkylimidazolinium salts of formulae XI or XII

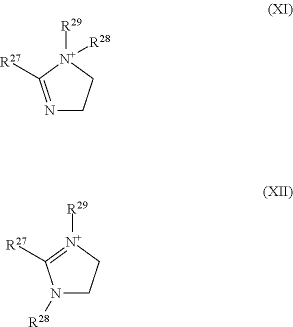

The variables in formulae (XI) and (XII) are defined as follows:

R²⁷ is selected from C₁-C₂₅-alkyl and C₂-C₂₅-alkenyl;

R²⁸ is selected from C₁-C₄-alkyl and hydroxy-C₁-C₄-alkyl;

R²⁹ is selected from C₁-C₄-alkyl, hydroxy-C₁-C₄-alkyl and a R*—(CO)—R³⁰—(CH₂)ⱼ— radical, wherein

R* is selected from C₁-C₂₁-alkyl and C₂-C₂₁-alkenyl; R³⁰ is selected from —O— and —NH—; j is 2 or 3.

In one embodiment, component C comprises two or more different cationic surfactants.

In one embodiment, component C comprises mixtures of at least one non-ionic and/or at least one amphoteric and/or at least one anionic surfactant and/or at least one cationic surfactant.

In one embodiment, at least one additional compound comprised in component C is selected from the group of foam-controlling substances. Foam-controlling substances include defoamers and foam stabilizers.

Non-limiting examples of suitable defoamers include alkyl phosphates, silicones and such as silicone emulsions (Wacker SRE-PFL, Silikon SRE, from Wacker Chemic, Germany or Rhodorsil from Rhodia, France), long-chain alcohols, fatty acids, salts of fatty acids, defoamers of the type of aqueous wax dispersions, solid defoamers (so-called compounds), organofluorine compounds, and mixtures thereof.

Suitable foam stabilizers include but are not limited to alkanolamides and alkylamine oxides.

In one embodiment, at least one additional compound comprised in component C is an antifreeze. An antifreeze usually lowers the freezing point of an aqueous liquid. Non-limiting examples of suitable antifreeze agents include liquid polyols, such as ethylene glycol, propylene glycol and glycerol.

In one embodiment, at least one additional compound comprised in component C is a rheology modifier. Rheology modifiers may be called structuring agents or structurants and may be selected from the following:

### i.) Polymeric Structuring Agents

Non-limiting examples of naturally derived polymeric structurants include hydroxyethyl cellulose, hydrophobically modified hydroxyethyl cellulose, carboxymethyl cellulose, polysaccharide derivatives, and mixtures thereof. Suitable polysaccharide derivatives include but are not limited to pectine, alginate, arabinogalactan (gum Arabic), carrageenan, gellan gum, xanthan gum, guar gum and mixtures thereof.

Non-limiting examples of synthetic polymeric structurants include: polycarboxylates, polyacrylates, hydrophobically modified ethoxylated urethanes, hydrophobically modified non-ionic polyols and mixtures thereof. A polycarboxylate polymer may for example be polyacrylate, polymethacrylate or mixtures thereof. The polyacrylate may be for example a copolymer of unsaturated mono- or di-carbonic acid and C₁-C₃₀ alkyl ester of the (meth)acrylic acid.

### ii.) Di-Benzylidene Polyol Acetal Derivative

A composition according to the invention may comprise one or more dibenzylidene polyol acetal derivatives (DBPA). The DBPA derivative may comprise a dibenzylidene sorbitol acetal derivative (DBS). Said DBS derivative may be selected from the group consisting of: 1,3:2,4-di-benzylidene sorbitol; 1,3:2,4-di(p-methylbenzylidene) sorbitol; 1,3:2,4-di(p-chlorobenzylidene) sorbitol; 1,3:2,4-di(2,4-dimethyldibenzylidene) sorbitol; 1,3:2,4-di (p-ethyl-benzylidene) sorbitol; 1,3:2,4-di(3,4-dimethyldibenzylidene) sorbitol; and mixtures thereof.

### iii.) Di-Amido-Gellants

In one aspect, the external structuring system may comprise a di-amido gellant having a molecular weight from about 150 g/mol to about 1,500 g/mol, or even from about 500 g/mol to about 900 g/mol. Such di-amido gellants may comprise at least two nitrogen atoms, wherein at least two of said nitrogen atoms form amido functional substitution groups. In one aspect, the amido groups are different. In another aspect, the amido functional groups are the same. The di-amido gellant has the following formula:

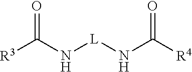

wherein the variables of the di-amido gellant in the above formula are defined as follows:

R³ and R⁴ is an amino functional end-group, or even amido functional end-group, in one aspect R³ and R⁴ may comprise a pH-tunable group, wherein the pH-tunable amido-gellant may have a pKa of from about 1 to about 30, or even from about 2 to about 10. In one aspect, the pH tunable group may comprise a pyridine. In one aspect, R³ and R⁴ may be different. In another aspect, R³ and R⁴ may be the same.

L is a linking moiety of molecular weight from 14 to 500 g/mol. In one aspect, L may comprise a carbon chain comprising between 2 and 20 carbon atoms. In another aspect, L may comprise a pH-tunable group. In one aspect, the pH-tunable group is a secondary amine. In one aspect, at least one of R³, R⁴ or L may comprise a pH-tunable group.

### iv.) Bacterial Cellulose

The term “bacterial cellulose” encompasses any type of cellulose produced via fermentation of a bacteria of the genus Acetobacter such as CELLULON® by CPKelco U.S. and includes materials referred to popularly as microfibrillated cellulose, reticulated bacterial cellulose, and the like.

In one aspect, said fibres may have cross sectional dimensions of 1.6 nm to 3.2 nm by 5.8 nm to 133 nm. Additionally, the bacterial cellulose fibres may have an average microfibre length of at least about 100 nm, or from about 100 to about 1,500 nm. In one aspect, the bacterial cellulose microfibres may have an aspect ratio, meaning the average microfibre length divided by the widest cross sectional microfibre width, of from about 100:1 to about 400:1, or even from about 200:1 to about 300:1.

In one aspect of the invention, the bacterial cellulose is at least partially coated with a polymeric structuring agents (see i. above). In one aspect, the at least partially coated bacterial cellulose comprises from about 0.1% to about 5% w/w, or even from about 0.5% to about 3% w/w of bacterial cellulose; and from about 10% to about 90% w/w of a polymeric structuring agent relative to the total weight of the liquid composition. Suitable bacterial cellulose may include the bacterial cellulose described above and suitable polymeric structuring agents include carboxymethylcellulose, cationic hydroxymethylcellulose, and mixtures thereof.

### v.) Cellulose Fibers Non-Bacterial Cellulose Derived

Cellulosic fibers may be extracted from vegetables, fruits or wood. Commercially available examples are Avicel® from FMC, Citri-Fi from Fiberstar or Betafib from Cosun.

### vi.) Non-Polymeric Crystalline Hydroxyl-Functional Materials

In one aspect of the invention, the composition may comprise non-polymeric crystalline, hydroxyl functional structurants. Said non-polymeric crystalline, hydroxyl functional structurants may comprise a crystallizable glyceride which can be pre-emulsified to aid dispersion into the liquid composition.

In one aspect, crystallizable glycerides may include hydrogenated castor oil or “HCO” or derivatives thereof, provided that it is capable of crystallizing in the liquid composition.

In one embodiment, component A remains dissolved in the liquid composition comprising components A, B, and C. Component A remains dissolved according to the invention, when no phase separation (precipitiation, flocculation, gelling turbitity) occurs due to the presence of component C.

The liquid composition of the invention may be a liquid detergent composition.

The liquid composition of the invention may be an additive in gasoline.

The invention provides a process for preparation of a liquid composition comprising the step of mixing in no specified order in one or more steps

component A: comprising at least one poly-lysine derivative,

component B: comprising a compound selected from the group of solvents in which component A is soluble, and

optionally component C: at least one additional compound,

wherein components A, B, and C are those disclosed above.

In one embodiment, the process for preparation of a liquid composition comprises the step of mixing in no specified order in one or more steps

component A: comprising at least one non-modified and/or modified poly-lysine derivative,

component B: comprising a compound selected from the group of solvents in which component A is soluble, and

optionally component C: at least one additional compound.

In one embodiment, the process for preparation of a liquid composition comprises the step of mixing in no specified order in one or more steps

component A: comprising at least one non-modified poly-lysine derivative which has been modified by alkoxylation such as ethoxylation and/or reaction with monofunctional molecules such as amines, isocyanate, carboxylic acids, alcohols such as mPEG, thiols, esters, acid chlorides, anhydrides, and carbonates,

component B: comprising a compound selected from the group of solvents in which component A is soluble, and

optionally component C: at least one additional compound.

The present invention provides a solid-based composition, comprising at least the following components:
1. dispersing medium: liquid composition of the invention comprising at least components A and B, and
2. component D—dispersed phase: at least one solid compound

wherein component D is insoluble in the dispersing medium.

In one embodiment, the dispersing medium comprises components A and B only.

In one embodiment, the dispersing medium comprises components A, B, and C.

Components A, B, and C are those disclosed above.

The solid compound comprised in component D includes any kind of particles of a size which are dispersible.

At least one solid compound means at least one type of solid compound. At least one solid compound of component D is solid at 20° C. and 101.3 kPa. In one embodiment, said solid compound is solid at 101.3 kPa and at a temperature of 40° C. In other embodiments said solid compound is solid at 101.3 kPa and at a temperature of 50° C., at a temperature of 60° C., at a temperature of 70° C., or at a temperature of 80° C.

In one embodiment, at least one solid compound of component D has a melting- or degradation point at 101.3 kPa which is above 20° C. In other embodiments, said solid compound has a melting- or degradation point at 101.3 kPa which is above 40° C., above 50° C., above 60° C., above 70° C., or 80° C. such as in the range of 80° C. to 300° C.

In one embodiment, at least one solid compound of component D is solid at 20° C. and 101.3 kPa and has a melting point of above 40° C.

In one embodiment, component D comprises two or more solid compounds.

At least one solid compound comprised in component D is insoluble in the dispersing medium, when the respective solid compound is soluble in the dispersing medium at 20° C. and 101.3 kPa in amounts less than 10% by weight, relative to the total amount of component D. At least one solid compound of component D may be insoluble in dispersing medium according to the invention, when the respective solid compound is soluble in the dispersing medium in amounts less than 5% by weight, in amounts less than 3% by weight, less than 1% by weight, or less than 0.5% by weight, all relative to the total amount of component D, all at 20° C. and 101.3 kPa.

At least one solid compound comprised in component D is insoluble in the dispersing medium, when the respective solid compound is soluble in 1000 g of the dispersing medium at 20° C. and 101.3 kPa in amounts less than 100 g. At least one solid compound of component D may be insoluble in the dispersing medium according to the invention, when the respective solid compound is soluble in 1000 g of the dispersing medium in amounts less than 50 g, in amounts less than 30 g, less than 10 g, or less than 5 g, all at 20° C. and 101.3 kPa.

In one embodiment, component D is insoluble in water at 20° C. and 101.3 kPa.

In one embodiment, at least one solid compound comprised in component D is selected from at least one agrochemically active compound, which may be selected from “pesticides” and “fertilizers”.

In one embodiment, the solid-based composition of the invention, comprising at least the following components:
1. dispersing medium: liquid composition of the invention comprising at least components A and B and optionally C, and
2. component D—dispersed phase: at least one solid compound selected from at least one agrochemically active compound

wherein at least one agrochemically active compound is insoluble in the dispersing medium, further comprises at least one agrochemically active compound which is soluble in the dispersing medium.

At least one agrochemically active compound comprised in the solid-based composition of the invention is soluble in the dispersing medium, when the respective agrochemically active compound is soluble in the dispersing medium until its saturation point in the dispersing medium at 20° C. and 101.3 kPa is achieved. The saturation point of at least one agrochemically active compound means the point (concentration) where the dispersing medium cannot dissolve any more of the substance at 20° C. and 101.3 kPa. Adding more than this maximum concentration of the agrochemically active compound will result in phase separation (precipitation, flocculation, gelling turbitity).

Pesticides may be selected from synthetic pesticides and biopesticides. The skilled worker is familiar with pesticides, which can be found, for example, in the Pesticide Manual, 17th Ed. (2015), The British Crop Protection Council, London. Non-limiting examples of pesticides include, but are not limited to fungicides, insecticides, nematicides, herbicides (algicides, arboricides, graminicides), akaricides, molluskicides, ovicides, rodenticides, safeners and growth regulators.

In one embodiment, component D comprises at least one solid pesticide selected from fungicides and/or insecticides and/or nematicides and/or herbicides and/or akaricides and/or molluskicides and/or ovicides and/or rodenticides and/or safeners and/or growth regulators. In one embodiment, component D comprises at least one solid pesticide selected from fungicides and/or insecticides and/or herbicides.

In one embodiment, component D comprises at least one solid fungicide and/or at least one solid insecticide and/or at least one solid nematicide and/or at least one solid herbicide and/or at least one solid akaricide and/or at least one solid molluskicide and/or at least one solid ovicide and/or at least one solid rodenticide and/or at least one solid safener and/or at least one solid growth regulator.

Non-limiting examples of suitable insecticides include compounds from the class of the carbamates, organophosphates, organochlorine insecticides, phenylpyrazoles, pyrethroids, neonicotinoids, spinosins, avermectins, milbemycins, juvenile hormone analogs, alkyl halides, organotin compounds nereistoxin analogs, benzoylureas, diacylhydrazines, METI acarizides, and insecticides such as chloropicrin, pymetrozin, flonicamid, clofentezin, hexythiazox, etoxazole, diafenthiuron, propargite, tetradifon, chlorofenapyr, DNOC, buprofezine, cyromazine, amitraz, hydramethylnon, acequinocyl, fluacrypyrim, rotenone, or their derivatives.

Non-limiting examples of suitable fungicides include compounds from the class of dinitroanilines, allylamines, anilinopyrimidines, antibiotics, aromatic hydrocarbons, benzenesulfonamides, benzimidazoles, benzisothiazoles, benzophenones, benzothiadiazoles, benzotriazines, benzyl carbamates, carbamates, carboxamides, carboxylic acid diamides, chloronitriles cyanoacetamide oximes, cyanoimidazoles, cyclopropanecarboxamides, dicarboximides, dihydrodioxazines, dinitrophenyl crotonates, dithiocarbamates, dithiolanes, ethylphosphonates, ethylaminothiazolecarboxamides, guanidines, hydroxy-(2-amino)pyrimidines, hydroxyanilides, imidazoles, imidazolinones, inorganic substances, isobenzofuranones, methoxyacrylates, methoxycarbamates, morpholines, N-phenylcarbamates, oxazolidinediones, oximinoacetates, oximinoacetamides, peptidylpyrimidine nucleosides, phenylacetamides, phenylamides, phenylpyrroles, phenylureas, phosphonates, phosphorothiolates, phthalamic acids, phthalimides, piperazines, piperidines, propionamides, pyridazinones, pyridines, pyridinylmethylbenzamides, pyrimidinamines, pyrimidines, pyrimidinonehydrazones, pyrroloquinolinones, quinazolinones, quinolines, quinones, sulfamides, sulfamoyltriazoles, thiazolecarboxamides, thiocarbamates, thiophanates, thiophenecarboxamides, toluamides, triphenyltin compounds, triazines, triazoles.

Non-limiting examples of suitable herbicides include compounds from the class of acetamides, amides, aryloxyphenoxypropionates, benzamides, benzofuran, benzoic acids, benzothiadiazinones, bipyridylium, carbamates, chloroacetamides, chlorocarboxylic acids, cyclohexanediones, dinitroanilines, dinitrophenol, diphenyl ether, glycines, imidazolinones, isoxazoles, isoxazolidinones, nitriles, N-phenylphthalimides, oxadiazoles, oxazolidinediones, oxyacetamides, phenoxycarboxylic acids, phenylcarbamates, phenylpyrazoles, phenylpyrazolines, phenylpyridazines, phosphinic acids, phosphoroamidates, phosphorodithioates, phthalamates, pyrazoles, pyridazinones, pyridines, pyridinecarboxylic acids, pyridinecarboxamides, pyrimidinediones, pyrimidinyl(thio)benzoates, quinolinecarboxylic acids, semicarbazones, sulfonylaminocarbonyltriazolinones, sulfonylureas, tetrazolinones, thiadiazoles, thiocarbamates, triazines, triazinones, triazoles, triazolinones, triazolocarboxamides, triazolopyrimidines, triketones, uracils, ureas.

Non-limiting examples of suitable growth regulators include abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N-6-benzyladenine, paclobutrazol, prohexadione (prohexadione-calcium), prohydrojasmon, thidiazuron, triapenthenol, tributyl phosphorotrithioate, 2,3,5-tri-iodobenzoic acid, triexapac-ethyl, and uniconazole.

“Safener” usually means compounds which are added to reduce or to avoid phytotoxic effects towards specific plants.

Fertilizer includes organic and synthetic fertilizers that may be applied to soils or plant tissue such as leaves to supply plant nutrients which usually enhance growth of plants. Fertilizers typically provide in varying proportions nitrogen and/or phosphorus and/or potassium and/or calcium and/or magnesium and/or sulfur and/or copper and/or iron and/or manganese and/or molybdenum and/or zinc and/or boron and/or other nutrients. Said nutrients may be provided as water-soluble salts.

Fertilizers may be provided in the form of controlled release fertilizers. For this purpose, fertilizers may be encapsuled in a shell that degrades as a specified rate, or fertilizers are provided in a granulated form from which the fertilizer leaches due to contact with water. At least one solid compound comprised in component D selected from fertilizers may therefore be an encapsuled fertilizer and/or a granulated fertilizer.

In one embodiment, component D comprises at least one solid pesticide and/or at least one solid fertilizer.

In one embodiment, solid-based compositions comprise two or more solid pesticides and/or two or more solid fertilizers.

In one embodiment, component D comprises at least one solid fungicide and/or at least one solid insecticide and/or at least one solid herbicide and/or at least one solid growth regulator and/or at least one solid fertilizer and at least one liquid fungicide and/or at least one liquid insecticide and/or at least one liquid herbicide and/or at least one liquid growth regulator and/or at least one liquid fertilizer.

Solid-based compositions of the invention comprising at least one solid pesticide and/or at least one solid fertilizer may be called solid-based agrochemical formulation herein.

The solid-based agrochemical formulation comprises at least one solid pesticide in amounts in the range of 0.1 to 80% by weight of relative to the total weight of the agrochemical formulation.

The solid-based agrochemical formulation may comprise at least one solid pesticide in amounts in the range of 0.1 to 75% by weight, or in the range of 1% to 75% by weight, all relative to the total weight of the agrochemical formulation.

The solid-based agrochemical formulation comprises a poly-lysine derivative according to the invention in amounts in the range of 0.1% to 40% by weight relative to the total weight of the agrochemical formulation. The solid-based agrochemical formulation may comprise a polylysine derivative according to the invention in amounts in the range of 0.1% to 30% by weight, in the range of 0.1% to 20% by weight, in the range of 0.1% to 15% by weight, or in the range of 0.1% to 10% by weight, all relative to the total weight of the agrochemical formulation.

In one embodiment, the solid based agrochemical composition comprises at least one non-modified and/or modified poly-lysine derivative. In one embodiment, the solid based agrochemical composition comprises at least one non-modified poly-lysine derivative which has been modified by alkoxylation such as ethoxylation and/or reaction with monofunctional molecules such as amines, isocyanate, carboxylic acids, alcohols such as mPEG, thiols, esters, acid chlorides, anhydrides, and carbonates.

In one embodiment, the solid based agrochemical composition comprises at least one non-modified and/or modified poly-lysine oleate. In one embodiment, the solid based agrochemical composition comprises at least one non-modified poly-lysine oleate which has been modified by alkoxylation such as ethoxylation and/or reaction with monofunctional molecules such as amines, isocyanate, carboxylic acids, alcohols such as mPEG, thiols, esters, acid chlorides, anhydrides, and carbonates.

In one embodiment, the solid based agrochemical composition comprises at least one non-modified and/or modified poly-lysine laurate. In one embodiment, component A comprises at least one non-modified poly-lysine laurate which has been modified by alkoxylation such as ethoxylation and/or reaction with monofunctional molecules such as amines, isocyanate, carboxylic acids, alcohols such as mPEG, thiols, esters, acid chlorides, anhydrides, and carbonates.

In one embodiment, the solid-based agrochemical formulation comprises water in amounts in the range of 1% to 99% by weight relative to the total weight of the agrochemical formulation.

The solid-based agrochemical formulation may comprise water in amounts in the range of 10% to 90% by weight, or in the range of 10% to 80% by weight, all relative to the total weight of the agrochemical formulation.

The agrochemical formulation of the invention may further comprise one or more formulation auxiliaries in amounts in the range of 0% to 80% by weight relative to the total weight of the agrochemical formulation. The solid-based agrochemical formulation may comprise one or more formulation auxiliaries in amounts in the range of 0% to 70% by weight, or in the range of 0% to 60% by weight, all relative to the total weight of the agrochemical formulation. Formulation auxiliaries are known to those skilled in the art and may be selected from surface-active substances (such as dispersants, emulsifiers, surfactants, solubilizers, protective colloids, wetters and stickers), solvents, solid carriers, defoamers, preservatives, antifreeze agents, rheology modifiers, colorants, antioxidants, retention enhancers (e.g. Lutensol® ON 60), penetration enhancers, adjuvants, tackifiers or binders (for example for the treatment of seeds) oils, and compatibilizer. The solid-based composition of the invention may comprise defoaming agents. Non-limiting examples of suitable, defoaming agents (also called defoamers) include silicone emulsions known for this purpose (Wacker SRE-PFL, Silikon SRE, from Wacker Chemic, Germany or Rhodorsil from Rhodia, France), long-chain alcohols, fatty acids, salts of fatty acids, defoamers of the type of aqueous wax dispersions, solid defoamers (so-called compounds), organofluorine compounds, and mixtures thereof. The amount of defoamers in a solid-based composition may be in the range of 0.01% to 1% by weight, in the range of 0.01% to 0.8% by weight, or in the range of 0.01% to 0.7% by weight, based on the total weight of the solid-based composition of the invention.

Besides at least one solvent comprised in the dispersing medium, the agrochemical formulation may comprise additional solvents (formulation auxiliary). Solvents may be selected from water, organic solvents such as mineral oil fractions of medium to high boiling point, coal tar oils and oils of vegetable or animal origin, aliphatic, cyclic and aromatic hydrocarbons (e.g. paraffins, tetrahydronaphthalene, alkylated naphthalenes and their derivatives, alkylated benzenes and their derivatives), alcohols, glycols, ketones, fatty acid dimethylamides, fatty acids and fatty acid esters and strongly polar solvents.

In one embodiment, at least one solid compound comprised in a solid-based agrochemical formulation of the invention is insoluble in the total amount of solvent comprised in the agrochemical formulation. At least one solid compound comprised in a solid-based agrochemical formulation of the invention is insoluble in the total amount of solvent comprised in the agrochemical formulation according to the invention, the respective solid compound is soluble in the total amount of solvents comprised in the agrochemical formulation at 20° C. and 101.3 kPa in amounts less than 10% by weight, relative to the total amount of component D. At least one solid compound of component D may be insoluble in the total amount of solvents comprised in the agrochemical formulation, when the respective solid compound is soluble in the total amount of solvents comprised in the agrochemical formulation in amounts less than 5% by weight, in amounts less than 3% by weight, or less than 1% by weight, all relative to the total amount of component D, all at 20° C. and 101.3 kPa. At least one solid compound comprised in a solid-based agrochemical formulation of the invention is insoluble in the total amount of solvent comprised in the agrochemical formulation according to the invention, when less than 100 g of the respective solid compound is soluble in 1000 g of solvents comprised in the agrochemical formulation at 20° C. and 101.3 kPa. At least one solid compound of component D may be insoluble in the total amount of solvents comprised in the agrochemical formulation, when less than 50 g, less than 30 g, or less than 1 g of the respective solid compound is soluble in 1000 g of solvents comprised in the agrochemical formulation at 20° C. and 101.3 kPa.

In one embodiment, component D comprised in a solid-based agrochemical formulation of the invention remains un-dissolved in the agrochemical formulation. Component D remains un-dissolved according to the invention, when at least 90% by weight of component D remains solid in the agrochemical formulation, relative to the total weight of component D. Component D also remains un-dissolved according to the invention, when at least 95% by weight, at least 97% by weight, at least 99% by weight, or at least 99.5% by weight of component D remains solid in the agrochemical formulation, relative to the total weight of component D.

In one embodiment, at least one solid compound comprised in component D is selected from at least one filling compound. Filling compound is a solid compound contributing texture of a solid based composition. Fillers are usually inert materials.

In one embodiment, at least one solid compound comprised in component D is selected from at least one pigment. Pigment is a solid compound usually contributing color. Pigments are selected from natural and synthetic pigments.

In one embodiment, at least one pigment is a hiding pigment. Hiding pigments may contribute opaqueness and/or UV protection.

In one embodiment, the solid-based composition of the invention is a painting composition.

In one embodiment, the solid-based composition of the invention is an ink.

In one embodiment, the solid-based composition of the invention is a paper coating.

In one embodiment, the solid-based compositions of the invention are stable during storage, meaning that neither significant increase in particle size of the dispersed solid compound (due to e.g. agglomeration), nor gelling, i.e. a significant increase in viscosity, is observed upon storage. Stability during storage herein may also mean that dispersed solid particles which have settled during storage are re-dispersible. Storage stability may be determined by storing a sample at 54° C. for 14 days (see e.g. CIPAC method MT 46—accelerated storage procedure) and comparing particle sizes before storage with particle sizes after storage.

The invention provides a process for preparation of a solid-based composition of the invention comprising the mixing in no specified order in one or more steps components A, B, optionally C and component D. In one embodiment, the process for preparation of a solid-based composition of the invention comprises the mixing of the liquid composition of the invention and component D. Components A, B, C and D are those disclosed above.

The invention provides a process for preparation of a solid-based agrochemical formulation comprising the mixing in no specified order in one or more steps components A, B, optionally C, component D, and at least one formulation auxiliary, wherein component A comprises at least one solid pesticide and/or at least one solid fertilizer. In one embodiment, the process for preparation of an agrochemical formulation of the invention comprises the mixing in no specified order in one or more steps components A, B, optionally C, component D, at least one liquid pesticide and/or liquid fertilizer, and at least one formulation auxiliary, wherein component A comprises at least one solid pesticide and/or at least one solid fertilizer. In one embodiment, the process for preparation of an agrochemical formulation of the invention comprises the mixing in no specified order in one or more steps of the liquid composition of the invention, component D, at least one liquid pesticide and/or liquid fertilizer, and at least one formulation auxiliary, wherein the liquid composition comprises at least one solid pesticide and/or at least one solid fertilizer. Components A, B, C, D, and formulation auxiliaries are those disclosed above.

The solid-based composition of the invention may be prepared by the process of comminution. Usually comminution processes divide a solid into fine particles in the dispersing medium or in a dry state before mixing with a dispersing medium. The one skilled in the art is familiar with the specifics of wet and dry comminution. The effectiveness of comminution depends on the shape and crystal form of particles. Usually, wet comminution is more effective than dry comminution and reduces particle size better. Wet comminution is often operated by using impeller mills, ball mills, small-media mills (such as sand mills and bead mills), vibratory mills, roll mills or ultrasonic dispersors. Further examples of mills useful include but are not limited to agitator ball mills, circulating mills (agitator ball mills with pin grinding system), disk mills, annular chamber mills, double cone mills, triple roll mills, batch mills, and colloid mills.

To dissipate the heat energy introduced during the comminution process, the comminution chambers may be fitted with cooling systems.

The particle size within 50% of the total amount of solid compound (dx₅₀) comprised in the solid-based composition of the invention may be about ≤50 μm, about ≤30 μm, about ≤20 μm, or about ≤10 μm.

In one embodiment, the particle size within 90% of the total amount of solid compound (dx₉₀) is less than 100 μm, less than 50 μm, less than 30 μm, or less than 20 μm.

Size particle distributions may be measured by any suitable method known to those skilled in the art. Suitable methods include but are not limited to methods using laser diffraction. Descriptions for the use of laser diffraction methods are provided e.g. in ISO 13320-1, CIPAC MT184 (Handbook K).

The invention provides the use of at least one poly-lysine derivative as wetting and/or dispersing agent for solid particles.

In one embodiment, at least one non-modified and/or modified poly-lysine derivative is used as wetting and/or dispersing agent for solid particles.

In one embodiment, at least one non-modified poly-lysine derivative at least one non-modified poly-lysine derivative which has been modified by alkoxylation such as ethoxylation and/or reaction with monofunctional molecules such as amines, isocyanate, isocyanate, carboxylic acids, alcohols such as mPEG, thiols, esters, acid chlorides, anhydrides, and carbonates is used as wetting and/or dispersing agent for solid particles.

In one embodiment, at least one poly-lysine derivative is used as wetting and/or dispersing agent for particles which are released during application of the composition of the invention.

In one embodiment, at least one non-modified and/or modified poly-lysine derivative is used as wetting and/or dispersing agent for solid particles which are released during application of the liquid composition of the invention.

In one embodiment, at least one non-modified poly-lysine derivative at least one non-modified poly-lysine derivative which has been modified by alkoxylation such as ethoxylation and/or reaction with monofunctional molecules such as amines, isocyanate, carboxylic acids, alcohols such as mPEG, thiols, esters, acid chlorides, anhydrides, and carbonates is used as wetting and/or dispersing agent for solid particles which are released during application of the liquid composition of the invention.

Release during application of the liquid composition of the invention includes but is not limited to cleaning application. Cleaning means any process directed to dissolution of particles and removal of the same.

In one embodiment, at least one poly-lysine derivative is used to prevent unwanted deposits in pipings.

In one embodiment, at least one non-modified and/or modified poly-lysine derivative is used to prevent unwanted deposits in pipings.

In one embodiment, at least one non-modified poly-lysine derivative at least one non-modified poly-lysine derivative which has been modified by alkoxylation such as ethoxylation and/or reaction with monofunctional molecules such as amines, isocyanate, carboxylic acids, alcohols such as mPEG, thiols, esters, acid chlorides, anhydrides, and carbonates is used to prevent unwanted deposits in pipings.

Unwanted deposits include but are not limited to microbial deposits and/or inorganic deposits and/or organic deposits.

Pipings include but are not limited to metallic and/or synthetic pipelines, plumbings, and tubings. In one embodiment, at least one poly-lysine derivative is used as wetting and/or dispersing agent in solid-based compositions.

In one embodiment, at least one non-modified and/or modified poly-lysine derivative is used as wetting and/or dispersing agent in solid-based compositions.

In one embodiment, at least one non-modified poly-lysine derivative at least one non-modified poly-lysine derivative which has been modified by alkoxylation such as ethoxylation and/or reaction with monofunctional molecules such as amines, isocyanate, carboxylic acids, alcohols such as mPEG, thiols, esters, acid chlorides, anhydrides, and carbonates is used as wetting and/or dispersing agent in solid-based compositions.

In one embodiment, the poly-lysine derivative is used as wetting and/or dispersing agent for solid particles during a comminution process.

In one embodiment, at least one non-modified and/or modified poly-lysine derivative is used as wetting and/or dispersing agent for solid particles during a comminution process.

In one embodiment, at least one non-modified poly-lysine derivative at least one non-modified poly-lysine derivative which has been modified by alkoxylation such as ethoxylation and/or reaction with monofunctional molecules such as amines, isocyanate, carboxylic acids, alcohols such as mPEG, thiols, esters, acid chlorides, anhydrides, and carbonates is used as wetting and/or dispersing agent for solid particles during a comminution process.

In one embodiment, the poly-lysine derivative is used to reduce redeposition of solid particles released during application.

In one embodiment, at least one non-modified and/or modified poly-lysine derivative is used to reduce redeposition of solid particles released during application of the composition.

In one embodiment, at least one non-modified poly-lysine derivative at least one non-modified poly-lysine derivative which has been modified by alkoxylation such as ethoxylation and/or reaction with monofunctional molecules such as amines, isocyanate, carboxylic acids, alcohols such as mPEG, thiols, esters, acid chlorides, anhydrides, and carbonates is used to reduce redeposition of solid particles released during application of the composition.

The current invention relates to the use of or method of use of at least one poly-lysine derivative to increase storage stability of solid-based compositions when compared to solid-based compositions lacking said poly-lysine derivative.

In one embodiment, at least one non-modified poly-lysine derivative and/or modified poly-lysine derivative is used to increase storage stability of solid-based compositions when compared to solid-based compositions lacking said non-modified poly-lysine derivative.

In one embodiment, at least one non-modified poly-lysine derivative which has been modified by alkoxylation such as ethoxylation and/or reaction with monofunctional molecules such as amines, isocyanate, carboxylic acids, alcohols such as mPEG, thiols, esters, acid chlorides, anhydrides, and carbonates is used to increase storage stability of solid-based compositions when compared to solid-based compositions lacking said non-modified poly-lysine derivative.

In one embodiment, poly-lysine oleate and/or poly-lysine laurate is used to increase storage stability of solid-based compositions when compared to solid-based compositions lacking said non-modified poly-lysine derivative.

The present invention provides the use or method of use of an agrochemical formulation of the invention for the treatment of plants. In one embodiment, an agrochemical formulation according to the invention is used for the treatment of crop plants.

Non-limiting examples of “crop plants”, such as cereals, e. g. wheat, rye, barley, triticale, oats or rice; beet, e. g. sugar beet or fodder beet; fruits, such as pomes, stone fruits or soft fruits, e. g. apples, pears, plums, peaches, almonds, cherries, strawberries, raspberries, blackberries or gooseberries; leguminous plants, such as lentils, peas, alfalfa or soybeans; oil plants, such as rape, mustard, olives, sun flowers, coconut, cocoa beans, castor oil plants, oil palms, ground nuts or soybeans; cucurbits, such as squashes, cucumber or melons; fiber plants, such as cotton, flax, hemp or jute; citrus fruit, such as oranges, lemons, grapefruits or mandarins; vegetables, such as spinach, lettuce, asparagus, cabbages, carrots, onions, tomatoes, potatoes, cucurbits or paprika; lauraceous plants, such as avocados, cinnamon or camphor; energy and raw material plants, such as corn, soybean, rape, sugar cane or oil palm; corn; tobacco; nuts; coffee; tea; bananas; vines (table grapes and grape juice grape vines); hop; turf; sweet leaf (also called Stevia); natural rubber plants or ornamental and forestry plants, such as flowers, shrubs, broad-leaved trees or evergreens, e. g. conifers; and on the plant propagation material, such as seeds, and the crop material of these plants.

The term “crop plant” is to be understood as including plants which have been modified by breeding, mutagenesis or genetic engineering including but not limiting to agricultural biotech products on the market or in development (cf. http://www.bio.org/speeches/pubs/er/agri products. asp). Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-translational modification of protein(s), oligo- or polypeptides e. g. by glycosylation or polymer additions such as prenylated, acetylated or farnesylated moieties or PEG moieties.

The use or method of use of agrochemical formulations of the invention may relate to the improvement of health of “crop plants” which may be determined by several indicators alone or in combination with each other such as yield (e. g. increased biomass and/or increased content of valuable ingredients), plant vigor (e. g. improved plant growth and/or greener leaves (“greening effect”)), quality (e. g. improved content or composition of certain ingredients) and tolerance to abiotic and/or biotic stress.

The use or method of use of agrochemical formulations of the invention may relate to the controlling of phytopathogenic fungi and/or undesired plant growth and/or undesired insect or mite attack and/or for regulating the growth of plants, where the agrochemical formulation of the invention is allowed to act on the respective pests, their environment or the plants to be protected from the respective pest, the soil and/or on undesired plants and/or the useful plants and/or their environment.

The invention provides the use or method of use of an agrochemical formulation according to the invention to treat plant propagation material.

The term “plant propagation material” is to be understood to denote all the generative parts of the plant such as seeds and vegetative plant material such as cuttings and tubers (e. g. potatoes), which can be used for the multiplication of the plant. This includes seeds, roots, fruits, tubers, bulbs, rhizomes, shoots, sprouts and other parts of plants, including seedlings and young plants, which are to be transplanted after germination or after emergence from soil.

These young plants may also be protected before transplantation by a total or partial treatment by immersion or pouring. In one embodiment, treatment of plant propagation materials with the composition and/or agrochemical formulation of the invention is used for controlling a multitude of fungi on cereals, such as wheat, rye, barley and oats; rice, corn, cotton and soybeans.

The invention relates to seed which has been treated with an agrochemical formulation of the invention. The seed may be dressed with the composition and/or agrochemical formulation of the invention. Dressing means that the seed is treated with the composition and or agrochemical formulation and the composition and/or agrochemical formulation remains on the seed. This composition and/or agrochemical composition may be applied to the seed in undiluted or, preferably, diluted form. Here, the composition in question can be diluted 2- to 10-fold, so that from 0.01% to 60% by weight, or from 0.1% to 40% by weight, of pesticide are present in the compositions and/or agrochemical formulation to be used for dressing the seed. The application can take place before sowing.

The treatment of plant propagation material, such as the treatment of seed, is known to the skilled worker and is carried out by dusting, coating, pelleting, dipping or soaking the plant propagation material, the treatment may be effected by pelleting, coating and dusting, so that, for example, premature germination of the seed is prevented. In the treatment of seed, one may use pesticide amounts of from 1 to 1000 g/100 kg, or from 5 to 100 g/100 kg propagation material or seed.

### EXAMPLES

### Example 1—General Process for Synthesis of Poly-Lysine Derivative

| | |
|---|---|
| Initial charge | 500 g Lysine solution 50% in water, 1.25 g |
| | sodium hypophosphite |
| feed 1: | 2000 g Lysine solution 50% in water |
| feed 2: | . . . g alkyl-carboxylic acid or alkenyl- |
| | carboxylic acid |

The initial charge is started to be heated. At an internal temperature of 100° C., feed 1 is started to be added to the boiling initial charge. After 45 minutes the internal temperature of 160° C. should be achieved. The internal temperature of the reaction mixture (i.e reaction temperature) is to be kept at this temperature at the following. Feed 1 is added within 5 hours to the reaction mixture.

After having added the whole feed 1, the pressure within the reaction system is to be reduced to 780 mbar within 35 minutes.

Within further 35 minutes, the pressure within the reaction system is to be further reduced to 725 mbar. The reaction mixture is to be kept at 160° C. and 725 mbar for additional 2 hours and 20 minutes.

During the whole time, evaporating water is distilled of.

The K-value is to be checked during the reaction several times. For this purpose, the vacuum is to be released to collect a sample and is to be applied again immediately after collecting the probe.

The K-value is to be determined by measurement of kinematic viscosity via Ubbelohde-viscosimeter (DIN 51562-3).

The amine number is to be checked after achieving the target K-value by potentiometric titration of the reaction mixture at 20° C. and 101.3 kPa with trifluoromethanesulfonic acid: amount of KOH in mg equals 1 g amine-comprising substance.

The molecular weight, viscosity and PDI are determined.

After reaching the target K-value and amine number, vacuum is to be released and feed 2 is to be added to the reaction mixture within 10 minutes.

Immediately after finishing the addition of feed 2, pressure within the reaction system is to be reduced to 725 mbar and the internal temperature of the reaction mixture is to be kept at 160° C. for another 4 hours. During this time, evaporating water is distilled of.

The weight-average molecular weight of the resulting poly-lysine derivative is to be determined by size exclusion chromatography (SEC or GPC) using hexafluoro iso-propanol with 0.055% of trifluoro acetic acid potassium salt as an eluent at 35° C. Signal calibration is done using a PMMA standard from the company PSS with molecular weights from 800 g/mol to 2,200,000 g/mol. Signal detection is performed by UV/Vis and refractive index sensors. Typically, 50 μL of sample having a concentration of 1.5 mg/mL are injected onto the column setup (1^{st} precolumn 8 mm inner diameter, 5 cm length; separation column one 7.5 mm inner diameter, 30 cm length; separation column two 7.5 mm inner diameter, 30 cm length) with a flow rate of 0.85 mL/min. Afterwards the internal pressure is to be set to atmospheric pressure and the temperature is to be reduced to 120° C. The product obtained is diluted with water to a concentration of about 30% and the pH is adjusted with lactic acid to a pH value of about 8.

### Example 2

| | |
|---|---|
| Initial charge | 500 g Lysine solution 50% in water, 1.25 g |
| | sodium hypophosphite |
| feed 1: | 2000 g Lysine solution 50% in water |
| feed 2: | 120.8 g Oleic acid |

The procedure described in example 1 was conducted until the poly-lysine reached a K value of 11; the poly-lysine had a Mw of 6,990 g/mol, Mn of 2,720 g/mol, and a PDI of 2.6. The amine number was 422, melt viscosity 3,280 mPa*s (measured with Epprecht viscosimeter at 140° C.), melt viscosity 1,000 mPa*s (measured with Epprecht viscosimeter at 160° C.).

Then feed 2 was introduced into the reaction mixture as described in example 1; the resulting poly-lysine oleate had a K-value of 14.9, an amine number of 315 mg KOH/g, Mw of 46,200 g/mol, Mn of 6,740 g/mol and a PDI of 6.9. Free acid was 2.1% relative to the total weight of the poly-lysine derivative (solid matter). The pH of the poly-lysine oleate solution was 8.3.

### Example 3

| | |
|---|---|
| Initial charge | 500 g Lysine solution 50% in water, 1.25 g |
| | sodium hypophosphite |
| feed 1: | 2000 g Lysine solution 50% in water |
| feed 2: | 120.8 g Oleic acid |

The procedure described in example 1 was conducted until the poly-lysine reached a K value of 12.3; the poly-lysine had a Mw of 17,100 g/mol, Mn of 4,910 g/mol, and a PDI of 3.5. The amine number was 391, melt viscosity 6,320 mPa*s (measured with Epprecht viscosimeter at 140° C.), melt viscosity 2,240 mPa*s (measured with Epprecht viscosimeter at 160° C.).

Then feed 2 was introduced as described in example 1; the resulting poly-lysine oleate had a K-value of 15.1, an amine number of 321 mg KOH/g, Mw of 49,700 g/mol, Mn of 7,420 g/mol and a PDI of 6.7. The pH of the poly-lysine oleate solution was 8.5.

### Example 4

| | |
|---|---|
| Initial charge | 500 g Lysine solution 50% in water, 1.25 g |
| | sodium hypophosphite |
| feed 1: | 2000 g Lysine solution 50% in water |
| feed 2: | 362.4 g Oleic acid |

The procedure described in example 1 was conducted until the poly-lysine reached a K value of 11; the poly-lysine had a Mw of 12,900 g/mol, Mn of 3,920 g/mol, and a PDI of 3.3. The amine number was 422, melt viscosity 3,280 mPa*s (measured with Epprecht viscosimeter at 140° C.), melt viscosity 1,000 mPa*s (measured with Epprecht viscosimeter at 160° C.).

Then feed 2 was introduced as described in example 1; the resulting poly-lysine oleate had an amine number of 221 mg KOH/g, Mw of 44,000 g/mol, Mn of 6,500 g/mol and a PDI of 6.8. Free acid was 2.4% relative to the total weight of the poly-lysine derivative (solid matter). The pH of the poly-lysine-oleate solution was 8.0.

### Example 5

| | |
|---|---|
| Initial charge | 500 g Lysine solution 50% in water, 1.25 g |
| | sodium hypophosphite |
| feed 1: | 2000 g Lysine solution 50% in water |
| feed 2: | 85.67 g Lauric acid |

The procedure described in example 1 was conducted until the poly-lysine reached a K-value of 12; the poly-lysine had a Mw of 22,700 g/mol, Mn of 5,850 g/mol, and a PDI of 3.9. The amine number was 391, melt viscosity 6,320 mPa*s (measured with Epprecht viscosimeter at 140° C.), melt viscosity 2,240 mPa*s (measured with Epprecht viscosimeter at 160° C.).

Then feed 2 was introduced into the reaction mixture as described in example 1; the resulting poly-lysine laurate had a K-value of 16.2, an amine number of 313 mg KOH/g, Mw of 81,400 g/mol, Mn of 9,340 g/mol and a PDI of 8.7. Free acid was 2.7% relative to the total weight of the poly-lysine derivative (solid matter). The pH of the poly-lysine laurate solution was 8.8.

### Example 6

| | |
|---|---|
| Initial charge | 500 g Lysine solution 50% in water, 1.25 g |
| | sodium hypophosphite, 212.5 g mPEG (Mw = |
| | 5000 g/mol, Pluriol A 5010E) |
| feed 1: | 2000 g Lysine solution 50% in water |
| feed 2: | 120.8 g Oleic acid |

The procedure described in example 1 was conducted until the poly-lysine reached a K-value of 12; the poly-lysine had a Mw of 13,900 g/mol, Mn of 3,000 g/mol, and a PDI of 4.7. The amine number was 395, melt viscosity 1,280 mPa*s (measured with Epprecht viscosimeter at 140° C.), melt viscosity 360 mPa*s (measured with Epprecht viscosimeter at 160° C.).

Then feed 2 was introduced into the reaction mixture as described in example 1; the resulting poly-lysine-oleate-mPEG had a K-value of 16.1, an amine number of 276 mg KOH/g, Mw of 34,400 g/mol, Mn of 7,450 g/mol and a PDI of 4.6. Free acid was 1.8% relative to the total weight of the poly-lysine derivative (solid matter). The pH of the poly-lysine-oleate-mPEG solution was 9.

### Example 7—Comparative Example: Synthesis of Poly-Lysine Oleate Based on Basodrill™ S 100

An aqueous solution of poly-lysine (9.934 kg) having a K value of 11 (Mw=17.100 g/mol, trade name Basodrill™ S100 by BASF) was dosed into the reactor. Successively water was removed from the solution at 160° C. Then oleic acid (Edenor™ TI05, 0.71 kg) was added to the reaction mixture and water was removed from the reaction mixture at 160° C. for 240 min. The reaction had to be stopped due to too high viscosity of the melt.

### Example 8: Dispersant Properties and Storage Stability of Solid-Based Composition

Composition: 50% azoxystrobin, 2.5% poly-lysine derivative, 0.3% defoamer, 47.2% water.

Control composition 1: 50% azoxystrobin, 2.5% Morwet® D425 (AkzoNobel, dispersant and wetting agent), 0.3% defoamer, 47.2% water.

Control composition 2: 50% azoxystrobin, 1.25% Morwet® D425 (AkzoNobel, dispersant and wetting agent), 2.5% Atlox™ 4913 (Croda; dispersant), 0.3% defoamer, 45.95% water.

Dispersant properties/storage stability in the solid-based composition:

| | | | | poly-lysine | poly-lysine | poly-lysine |
|---|---|---|---|---|---|---|
| | | | | oleate of | oleate of | laurate of |
| | | Control 1 | Control 2 | Example 3 | Example 4 | Example 5 |
| START | dx₁₀ | 0.69 μm | 0.64 μm | 0.68 μm | 0.73 μm | 0.71 μm |
| | dx₅₀ | 1.54 μm | 1.42 μm | 1.67 μm | 1.74 μm | 1.77 μm |
| | dx₉₀ | 3.12 μm | 2.58 μm | 3.48 μm | 3.51 μm | 3.74 μm |
| After | dx₁₀ | 0.73 μm | 0.70 μm | 0.71 μm | 0.68 μm | 0.73 μm |
| storage | dx₅₀ | 2.00 μm | 1.76 μm | 1.80 μm | 1.80 μm | 1.92 μm |
| | dx₉₀ | 4.44 μm | 4.19 μm | 4.04 μm | 3.90 μm | 4.43 μm |

The “START” value provides data for a solid-based composition directly after preparation of the same wet comminution.
The “after storage” value provides data for a solid-based composition after storage of the start solid-based composition at 54° C. for 14 days.

1. A poly-lysine derivative obtained by a process comprising the steps of:
(a) heating an aqueous lysine solution to boiling;
(b) increasing a temperature of the aqueous lysine solution to a reaction temperature in the range of about 105° C. to about 180° C.;
(c) keeping the reaction temperature in the range of about 105° C. to about 180° C. until:
i. a melt viscosity of the reaction mixture is in the range of about 350 mPa*s to about 6,500 mPa*s when measured at 160° C.; and
ii. an amine number in the range of about 100 mg KOH/g to about 500 mg KOH/g is achieved;
(d) optionally, releasing a vacuum applied;
(e) adding alkyl-carboxylic acid or alkenyl-carboxylic acid in amounts of 2.5 mol % to 10 mol %, relative to a theoretical amount of poly-lysine in the reaction mixture; and
(f) increasing or keeping the reaction temperature in the range of about 105° C. to about 180° C. until number of free alkyl-carboxylic acid or alkenyl-carboxylic acid is ≤9% by weight, relative to the total weight of the reaction mixture,
wherein vacuum is applied in step (a), (b), and/or (c) and water is removed continuously during the process.

2. The poly-lysine derivative obtained by a process according to claim 1, wherein the poly-lysine derivative obtained is further processed by the following additional steps:
(g) vacuum applied is released and the temperature within the reaction mixture is reduced to about 150° C. to 100° C.; and
(h) water is added to yield a solution comprising about 60 parts of poly-lysine derivative and about 40 parts of water.

3. A non-crosslinked poly-lysine oleate, wherein the poly-lysine oleate has a weight-average molecular weight in the range of about 30,000 g/mol to about 50,000 g/mol.

4. The non-crosslinked poly-lysine oleate according to claim 3, wherein the poly-lysine oleate has a polydispersity index of about 4.0 to about 9.0.

5. The non-crosslinked poly-lysine oleate according to claim 3, wherein the non-crosslinked poly-lysine oleate is water-soluble.

6. A process for preparation of poly-lysine derivative, which comprises the steps of:
(a) heating an aqueous lysine solution to boiling;
(b) increasing a temperature of the aqueous lysine solution to a reaction temperature in the range of about 105° C. to about 180° C.;
(c) keeping the reaction temperature in the range of about 105° C. to about 180° C. until;
i. a melt viscosity of the reaction mixture is in the range of about 350 mPa*s to about 6,500 mPa*s when measured at 160° C.; and
ii. an amine number in the range of about 100 mg KOH/g to about 500 mg KOH/g is achieved;
(d) optionally, releasing a vacuum applied;
(e) adding alkyl-carboxylic acid or alkenyl-carboxylic acid in amounts of 2.5 mol % to 10 mol %, relative to a theoretical amount of poly-lysine in the reaction mixture; and
(f) increasing or keeping the reaction temperature in the range of about 105° C. to about 180° C. until number of free alkyl-carboxylic acid or alkenyl-carboxylic acid is ≤9% by weight, relative to the total weight of the reaction mixture,
wherein vacuum is applied in step (a), (b) and/or (c), and water is removed continuously during the process.

7. A liquid composition comprising at least:
component A: comprising at least one poly-lysine derivative according to claim 1,
component B: comprising a compound selected from the group of solvents in which component A is soluble, and
optionally component C: at least one additional compound.

8. The liquid composition according to claim 7, wherein at least one additional compound of component C is selected from the group of surfactants.

9. A process for preparation of a liquid composition according to claim 7, the process comprising mixing in no specified order in one or more steps component A, component B, and optionally component C.

10. A solid-based composition comprising:
a dispersing medium comprising the liquid composition according to claim 7 comprising at least components A and B, and
component D: at least one solid compound,
wherein the at least one solid compound is solid at 20° C. and 101.3 kPa and insoluble in the dispersing medium.

11. A solid-based composition according to claim 10, wherein the at least one solid compound of component D is selected from agrochemically active compounds.

12. A process for preparation of a solid-based composition according to claim 10, the process comprising mixing in no specified order in one or more steps component A, component B, optionally component C, and component D.

13. The process for preparation of a solid-based composition according to claim 12, wherein the process comprising comminuting at least one of component A, component B, optional component C, and component D.

14. A method of using at least one poly-lysine derivative according to claim 1, wherein the method comprises using the poly-lysine derivative as a wetting and/or dispersing agent for solid particles.

15. A method of using at least one poly-lysine derivative according to claim 1, wherein the method comprises using the poly-lysine derivative to increase storage stability of solid based compositions, when compared to solid-based compositions lacking poly-lysine derivative.
